(19) United States
(12) Patent Application Publication
(10) Pub. No.: US 2018/0078609 A1
(43) Pub. Date: Mar. 22, 2018

(54) METHODS AND COMPOSITIONS FOR TREATING ALZHEIMER'S DISEASE AND OTHER MEMORY-ASSOCIATED DISORDERS AND CONDITIONS

(71) Applicant: Massachusetts Institute of Technology, Cambridge, MA (US)

(72) Inventors: Susumu Tonegawa, Newton, MA (US); Dheeraj Roy, Watertown, MA (US)

(73) Assignee: Massachusetts Institute of Technology, Cambridge, MA (US)

(21) Appl. No.: 15/272,969

(22) Filed: Sep. 22, 2016

(51) Int. Cl. A61K 38/17; A61N 5/06

(52) U.S. Cl. CPC A61K 38/1709; A61N 2005/0663; A61N 5/062

(57) ABSTRACT

The invention, in part, relates to the use of optogenetic methods to increase dendritic spine density on DG memory engram cells in treatment methods for memory-impairment-associated diseases and conditions.

### FIELD OF THE INVENTION

This application relates, in part, to mechanisms of memory retrieval and storage in early Alzheimer's disease and methods to treat memory-impairment diseases and additional conditions.

### BACKGROUND OF THE INVENTION

Alzheimer's Disease (AD) is the most common cause of brain degeneration, and typically begins with impairments in cognitive functions [Selkoe, D. J. (2001) Physiol. Rev. 81, 741-766]. Most research has focused on understanding the relationship between memory impairments and the formation of two pathological hallmarks seen in the late stages of AD: extracellular amyloid plaques and intracellular aggregates of tau protein [Selkoe, D. J. (2001) Physiol. Rev. 81, 741-766] [Selkoe, D. J. (2002) Science 298, 789-791]. The early phases of AD have received relatively less attention, although synaptic phenotypes have been identified as major correlates of cognitive impairments in both human patients and mouse models [Jacobsen, J. S., et al. (2006) Proc. Natl. Acad. Sci. USA 103, 5161-5166] [Terry, R. D., et al. (1991) 30, 572-580]. Several studies have suggested that the episodic memory deficit of AD patients is due to ineffective encoding of new information [Granholm, E. & Butters, N. (1988) Brain Cogn. 7, 335-347][Hodges, J. R. et al (1990) 53, 1089-1095] [Weintraub, S. et al. (2012) Cold Spring Harb. Perspect. Med. 2, a006171]. However, since the cognitive measures used in these studies rely on memory retrieval, it is not possible to discriminate rigorously between impairments in information storage and disrupted retrieval of stored information. This issue has an important clinical implication: if the amnesia is due to retrieval impairments, memory could be restored by technologies involving targeted brain stimulation.

### SUMMARY OF THE INVENTION

According to an aspect of the invention, methods of selectively producing one or both of a temporally sustained stabilization of, and increase in, dendritic spine density in one or a plurality of dentate gyrus (DG) memory engram cells in a subject are provided. The methods including: selectively inducing high-frequency neuronal firing in one or a plurality of DG memory engram cells in the subject; in an amount effective for one or both of stabilizing and increasing dendritic spine density in the one or the plurality of DG memory engram cells, wherein the stability of, and increase in, dendritic spine density is temporally sustained. In certain embodiments, a means for selectively inducing the high-frequency neuronal firing includes: (a) expressing in one or a plurality of first cells in the subject, a stimulus-activated opsin polypeptide; wherein activating the expressed stimulus-activated opsin polypeptide selectively induces high-frequency neuronal firing in the one or the plurality of DG memory engram cells in the subject; and (b) stimulating the stimulus-activated opsin polypeptide a first time under conditions suitable to selectively induce the high-frequency neuronal firing in the one or the plurality of DG memory engram cells, wherein the induced high-frequency neuronal firing stabilizes or increases the temporally sustained dendritic spine density in the one or the plurality of DG memory engram cells. In some embodiments, the method also includes: (c) stimulating the stimulus-activated opsin polypeptide one or more subsequent times each time under an independently selected stimulation condition of one or: (i) the same as set forth in an aforementioned embodiment (b) and (ii) different from those set forth in the aforementioned embodiment (b), wherein the selected stimulation condition is suitable to selectively induce the high-frequency neuronal firing in the one or the plurality of DG memory engram cells, the induced high-frequency neuronal firing results in one or both of: stabilizing and increasing the temporally sustained dendritic spine density in the one or more DG memory engram cells. In some embodiments, an interval of time between the end of a first stimulation period of the stimulus-activated opsin polypeptide and start of a subsequent stimulation period of the stimulus-activated opsin polypeptide is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, and 20 weeks. In certain embodiments, an interval of time between the end of a first or subsequent stimulation period of the stimulus-activated opsin polypeptide and start of another subsequent stimulation period of the stimulus-activated opsin polypeptide is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, and 20 weeks. In some embodiments, the frequency of the induced high-frequency neuronal firing is at least 45 Hz, 50 Hz, 60 Hz, 70 Hz, 80 Hz, 90 Hz, 100 Hz, or 110 Hz. In certain embodiments, the time duration of the stimulation period of the stimulus-activated opsin polypeptide is one or more of: between 1 and 3 hours, between 3 and 7 hours, between 0.5 and 7 hours, between 0.1 and 10 hours, between 0.1 and 24 hours, between 0.1 and 24 hours, between 0.1 and 36 hours, between 0.1 and 48 hours, between 0.1 and 60 hours. In some embodiments, the time duration of the stimulation period of the stimulus-activated opsin polypeptide is at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 50, 60, 70, 80, 90, and 100 hours. In certain embodiments, the frequency of the induced high-frequency neuronal firing is at least 90 Hz and the duration of the stimulation period is between 1 and 3 hours. In some embodiments, the frequency of the induced high-frequency neuronal firing is at least 50 Hz and the duration of the stimulation period is between 3 and 7 hours. In some embodiments, to increase the dendritic spine density in a DG memory engram cell of the subject, the frequency of the induced high-frequency neuronal firing is at least 90 Hz and the duration of the stimulation period is at least one hour. In certain embodiments, to stabilize the dendritic spine density in a DG memory engram cell of the subject, the frequency of the induced high-frequency neuronal firing is at least 50 Hz and the duration of the stimulation period is at least one hour. In some embodiments, the stimulus-activated opsin polypeptide is a light-activated opsin polypeptide. In some embodiments, the stimulus-activated opsin polypeptide comprises a ChIEF polypeptide or a functional variant thereof. In certain embodiments, the stimulus-activated opsin polypeptide comprises a ChrimsonR, Chronos, oChIEF, or ChEF polypeptide or a functional variant thereof. In some embodiments, the first cell is an entorhinal cortex (EC) cell or a DG cell. In some embodiments, the first cell projects to at least one DG memory engram cell in the subject. In some embodiments, the stimulus-activated opsin polypeptide is expressed as part of a fusion protein. In certain embodiments, the method also includes, delivering the stimulus-activated opsin polypeptide to the first cell in the form of a fusion protein comprising the stimulus-activated opsin polypeptide, or in the form of a vector comprising a nucleic acid sequence encoding the stimulus-activated opsin polypeptide. In some embodiments, the fusion protein additionally includes one or more of: a: trafficking agent polypeptide, targeting agent polypeptide, and a detectable-label polypeptide. In certain embodiments, the vector also includes a nucleic acid sequence encoding one or more of a: trafficking agent polypeptide, targeting agent polypeptide, and a detectable-label polypeptide. In some embodiments, the selective induction of the high-frequency neuronal firing in the one or more DG memory engram cells increases the synaptic connectivity between one or more entorhinal cortex (EC) cells and one or more of the DG memory engram cells in the subject. In certain embodiments, the selective induction of the high-frequency neuronal firing is restricted to one or more of: the at least one first cell, one or more cells directly downstream from the at least one first cell, and one or more cells indirectly downstream from the at least one first cell. In some embodiments, the temporally sustained increase in dendritic spine density in one or more dentate gyrus (DG) memory engram cells in the subject, results in at least one of: increasing, maintaining, and slowing a reduction of a level of memory retrieval in the subject. In some embodiments, the temporally sustained stabilization in dendritic spine density in one or more dentate gyrus (DG) memory engram cells in the subject, results in at least one of: increasing, maintaining, and slowing a reduction of a level of memory retrieval in the subject. In certain embodiments, one or both of temporally sustained stabilizing and increasing dendritic spine density in the one or the plurality of DG memory engram cells in the subject treats a memory impairment-associated disease or condition in the subject. In some embodiments, treating the memory-impairment-associated disease or condition comprises one or more of: an increase in memory-retrieval ability in the subject, a slowing of a reduction in memory-retrieval ability in the subject, a stopping of a reduction in memory retrieval-ability in the subject, and a reversal of a reduction in memory-retrieval ability in the subject. In some embodiments, the subject is at least one of: suspected of having, at risk of having, and diagnosed with a memory-impairment-associated disease or condition. In certain embodiments, the memory-impairment-associated disease or condition is one or more of: dementia, memory loss, brain injury, senility, a learning deficit, a memory deficit, an autoimmune disease, Huntington's disease, a degenerative neurological disease, amnesia, and Alzheimer's disease. In some embodiments, the memory-impairment associated disease is an early-stage memory-impairment associated disease. In certain embodiments, the memory-impairment associated disease is a mid-stage memory-impairment associated disease, and in some embodiments the memory-impairment associated disease is a late-stage memory-impairment associated disease. In some embodiments, the means for determining the stage of the memory-impairment associated disease or condition as one of early stage, mid-stage, and late-stage includes use of diagnostic and assessment methods. In some embodiments, the subject is one or more of: at elevated risk of having Alzheimer's disease, suspected of having Alzheimer's disease, and diagnosed with Alzheimer's disease. In certain embodiments, the Alzheimer's disease is early stage Alzheimer's disease. In certain embodiments, the Alzheimer's disease is mid-stage Alzheimer's disease, and in some embodiments the Alzheimer's disease is late-stage Alzheimer's disease. In certain embodiments, subject is one or more of: not diagnosed with and not suspected of having a reduced level of memory retrieval. In some embodiments, the subject does not have and is not suspected of having one or more of: dementia, memory loss, brain injury, senility, a learning deficit, a memory deficit, an autoimmune disease, a degenerative neurological disease, amnesia, and Alzheimer's disease. In some embodiments, the stimulus comprises illumination. In certain embodiments, the illumination characteristics include one or more of: a wavelength of the illumination, a time period of the illumination, a time interval between two or more illumination periods, a pulse frequency of the illumination, and an intensity of the illumination. In some embodiments, the stimulation is one of chronic stimulation and acute stimulation. In some embodiments, the method additionally including: modifying one or more additional treatments to the subject to treat or assist in treating the memory-impairment-associated disease or condition. In certain embodiments, modifying an additional treatment comprises at least one of: one or more of: starting, maintaining, increasing, reducing, or stopping administration of an additional therapeutic agent to the subject; one or more of: starting, maintaining, increasing, reducing, or stopping administration of a behavioral therapy to the subject; one or more of starting, maintaining, increasing, reducing, or stopping administration of a deep brain stimulation therapy to the subject; administering a surgical therapy to the subject; one or more of starting, maintaining, increasing, reducing, and stopping administering a cognitive therapy to the subject; and one or more of: starting, maintaining, increasing, reducing and stopping administering a counseling therapy to the subject. In some embodiments, wherein the therapeutic agent is one or more of a cholinesterase inhibitor, memantine, an antidepressant, an anxiolytic, and an antipsychotic. In some embodiments, a means of selectively inducing high-frequency neuronal firing in one or more dentate gyrus (DG) memory engram cells does not include one or more of: deep brain stimulation and pharmacological induction. In certain embodiments, a means of producing a temporally sustained increase in dendritic spine density in one or more dentate gyrus (DG) memory engram cells does not include one or more of: deep brain stimulation and a pharmacological treatment. In some embodiments, inducing high-frequency neuronal firing includes inducing long-term potentiation in the DG memory engram cells. In some embodiments, the subject is a mammal. In some embodiments, the subject is a human. In certain embodiments, the subject is engaged in learning during at least a portion of the high-frequency neuronal firing. In some embodiments, the stimulus-activated opsin polypeptide is not a ChR2 polypeptide or a variant thereof that is not capable of inducing high frequency firing in a memory engram cell when used in a method of an aforementioned embodiment or aspect of the invention.

In another aspect of the invention, methods of treating memory-impairment-associated disease or condition in a subject are provided, the methods including: (a) expressing in a first cell in a subject in need of such treatment, an effective amount of a stimulus-activated opsin polypeptide; wherein activating the expressed stimulus-activated opsin polypeptide selectively induces high-frequency neuronal firing in one or more DG memory engram cells in the subject; and (b) stimulating the stimulus-activated opsin polypeptide a first time under conditions suitable to selectively induce the high-frequency neuronal firing in the one or more DG memory engram cells, wherein the induced high-frequency neuronal firing increases a level of density of dendritic spines of the one or more DG memory engram cells; the increased density is temporally sustained in the subject; and the increased density of the dendritic spines treats the memory-impairment-associated disease or condition in the subject. In certain embodiments, the method also includes (c) stimulating the stimulus-activated opsin polypeptide one or more subsequent times each time under one of: (i) the stimulation conditions of (b) and (ii) stimulation conditions different from (b) suitable to selectively induce the high-frequency neuronal firing in the one or more DG memory engram cells, wherein the induced high-frequency neuronal firing results in one or both of: increasing the temporally sustained dendritic spine density in the one or more DG memory engram cells and maintaining the temporally sustained dendritic spine density increased in (b), in the one or more DG memory engram cells. In some embodiments, the end of a stimulation period of the first stimulation of the stimulus-activated opsin polypeptide and start of a subsequent stimulation period of the stimulus-activated opsin polypeptide is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, and 20 weeks. In some embodiments, an interval of time between the end of a first or subsequent stimulation period of the stimulus-activated opsin polypeptide and start of another subsequent stimulation period of the stimulus-activated opsin polypeptide is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, and 20 weeks. In certain embodiments, the frequency of the induced high-frequency neuronal firing is at least 45 Hz, 50 Hz, 60 Hz, 70 Hz, 80 Hz, 90 Hz, 100 Hz, or 110 Hz. In some embodiments, the time duration of the stimulation period of the stimulus-activated opsin polypeptide is one or more of: between 1 and 3 hours, between 3 and 7 hours, between 0.5 and 7 hours, between 0.1 and 10 hours, between 0.1 and 24 hours, between 0.1 and 24 hours, between 0.1 and 36 hours, between 0.1 and 48 hours, between 0.1 and 60 hours. In certain embodiments, the time duration of the stimulation period of the stimulus-activated opsin polypeptide is at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 50, 60, 70, 80, 90, and 100 hours. In some embodiments, the frequency of the induced high-frequency neuronal firing is at least 90 Hz and the duration of the stimulation period is between 1 and 3 hours. In some embodiments, the frequency of the induced high-frequency neuronal firing is at least 50 Hz and the duration of the stimulation period is between 3 and 7 hours. In certain embodiments, the frequency and duration of the induced high-frequency neuronal firing to increase the dendritic spine density are at least 90 Hz and at least one hour, respectively. In some embodiments, the frequency and duration of the induced high-frequency neuronal firing to maintain the dendritic spine density increased are at least 50 Hz and at least one hour, respectively. In some embodiments, the stimulus-activated opsin polypeptide is a light-activated opsin polypeptide. In certain embodiments, the stimulus-activated opsin polypeptide is a ChrimsonR, Chronos, ChEF, oChIEF, or ChIEF polypeptide or a functional variant thereof. In some embodiments, the stimulus-activated opsin polypeptide is a ChIEF polypeptide or a functional variant thereof. In some embodiments, the first cell is an entorhinal cortex (EC) cell or a DG cell. In certain embodiments, the first cell projects to at least one DG memory engram cell in the subject. In some embodiments, the stimulus-activated opsin polypeptide is expressed as part of a fusion protein. In some embodiments, the method also includes delivering the stimulus-activated opsin polypeptide to the first cell in the form of a fusion protein comprising the stimulus-activated opsin polypeptide, or in the form of a vector comprising a nucleic acid sequence encoding the stimulus-activated opsin polypeptide. In some embodiments, the fusion protein further includes one or more of: a: trafficking agent polypeptide, targeting agent polypeptide, and a detectable-label polypeptide. In certain embodiments, the vector also includes a nucleic acid sequence encoding one or more of a: trafficking agent polypeptide, targeting agent polypeptide, and a detectable-label polypeptide. In some embodiments, the selective induction of the high-frequency neuronal firing in the one or more DG memory engram cells increases the synaptic connectivity between one or more entorhinal cortex (EC) cells and one or more of the DG memory engram cells in the subject. In some embodiments, the selective induction of the high-frequency neuronal firing is restricted to one or more of: the at least one first cell, one or more cells directly downstream from the at least one first cell, and one or more cells indirectly downstream from the at least one first cell. In certain embodiments, the temporally sustained increase in dendritic spine density in one or more dentate gyrus (DG) memory engram cells in the subject, increases a level of memory retrieval in the subject. In some embodiments, treating the memory-impairment-associated disease or condition comprises one or more of: increasing memory retrieval in the subject, slowing a reduction in memory retrieval in the subject, stopping a reduction in memory retrieval in the subject, and reversing a reduction in memory retrieval in the subject. In some embodiments, the subject is at least one of: suspected of having, at risk of having, and has been diagnosed as having a memory-impairment-associated disease or condition. In certain embodiments, the memory-impairment-associated disease or condition is one or more of: dementia, memory loss, brain injury, senility, a learning deficit, a memory deficit, an autoimmune disease, a degenerative neurological disease, and Alzheimer's disease. In some embodiments, the memory-impairment associated disease is an early-stage memory-impairment associated disease. In certain embodiments, the memory-impairment associated disease is a mid-stage memory-impairment associated disease, and in some embodiments the memory-impairment associated disease is a late-stage memory-impairment associated disease. In some embodiments, the means for determining the stage of the memory-impairment associated disease or condition as one of early stage, mid-stage, and late-stage includes use of diagnostic and assessment methods. In some embodiments, the subject is one or more of: at risk of having Alzheimer's disease, suspected of having Alzheimer's disease, and diagnosed as having Alzheimer's disease. In certain embodiments, the Alzheimer's disease is early stage Alzheimer's disease. In certain embodiments, the Alzheimer's disease is mid-stage Alzheimer's disease, and in some embodiments the Alzheimer's disease is late-stage Alzheimer's disease. In some embodiments, the stimulus includes illumination. In certain embodiments, a characteristic of the illumination includes one or more of: a wavelength of the illumination, a time period of the illumination, a frequency of two or more periods of illumination, an interval between two or more illumination periods, and an intensity of the illumination. In some embodiments, the stimulation is chronic stimulation. In some embodiments, the stimulation is acute stimulation. In certain embodiments, the method also includes altering one or more additional treatments administered to the subject to treat or assist in treating the memory-impairment-associated disease or condition. In some embodiments, modifying an additional treatment includes at least one of: one or more of: starting, maintaining, increasing, reducing, or stopping administration of an additional therapeutic agent to the subject; one or more of: starting, maintaining, increasing, reducing, or stopping administration of a behavioral therapy to the subject; one or more of starting, maintaining, increasing, reducing, or stopping administration of a deep brain stimulation therapy to the subject; administering a surgical therapy to the subject; one or more of starting, maintaining, increasing, reducing, and stopping administering a cognitive therapy to the subject; and one or more of: starting, maintaining, increasing, reducing and stopping administering a counseling therapy to the subject. In some embodiments, the additional therapeutic agent is one or more of a cholinesterase inhibitor, memantine, an antidepressant, an anxiolytic, and an antipsychotic. In certain embodiments, a means of selectively inducing high-frequency neuronal firing in one or more dentate gyrus (DG) memory engram cells does not include one or more of: deep brain stimulation and pharmacological induction. In some embodiments, the subject is engaged in learning during at least a portion of the high-frequency neuronal firing. In some embodiments, the stimulus-activated opsin polypeptide is not a ChR2 polypeptide or a variant thereof that is not capable of inducing high frequency firing in a memory engram cell when used in a method of an aforementioned embodiment or aspect of the invention.

### BRIEF DESCRIPTION OF THE DRAWINGS

FIG. 1A-X shows optogenetic activation of memory engrams restores fear memory in early AD mice. FIG. 1A-C shows amyloid-β (Aβ) plaques in 9-month old AD mice. FIG. 1A is a lower-power view of FIGS. 1B & C. FIG. 1B highlights plaques in the dentate gyrus (DG) and FIG. 1C highlights plaques in the entorhinal cortex (EC). FIG. 1D is a graph showing plaque counts in hippocampal (HPC) sections (n=4 mice per group and “ND” is not detected). FIG. 1E shows a contextual fear conditioning (CFC) behavioral schedule (n=10 mice per group). FIG. 1F-I provides graphs of: freezing levels of 7-month old AD groups during training in FIG. 1F; short-term memory (STM) test results in FIG. 1G; long-term memory (LTM) test results in FIG. 1H, and results from exposure to neutral context in FIG. 1I. FIG. 1J is a graph showing c-Fos⁺ cell counts in the DG of 7-month-old mice after CFC training or LTM test as represented in FIGS. 1F and H (n=4 mice per group); “DAPI” is 4′,6-diamidino-2-phenylindole treatment. FIG. 1K-N provides graphs of: freezing levels of 9-month-old AD mice during training (FIG. 1K), STM test (FIG. 1L), LTM test (FIG. 1M), or exposure to neutral context (FIG. 1N). FIG. 1O is a graph of c-Fos⁺ cell counts in the DG of 9-month-old mice (n=3 mice per group) after CFC training represented in FIG. 1K. FIG. 1P provides a schematic diagram of an embodiment of a virus-mediated engram labelling strategy using a cocktail of AAV₉-c-Fos.tTA and AAV₉-TRE-ChR2-eYFP. FIG. 1Q is a schematic diagram showing an embodiment of treatment in which AD mice were injected with the two viruses bilaterally and implanted with an optic fiber bilaterally into the DG. FIG. 1R shows embodiment of behavioral schedule and photomicrographs showing DG engram cell labelling. FIG. 1S is a graph of ChR2-eYFP⁺ cell counts from DG sections shown in FIG. 1R (n=3 mice per group). FIG. 1T shows an embodiment of a behavioral schedule for optogenetic activation of DG engram cells. FIG. 1U provides a graph of ChR2-eYFP⁺ cell counts from 7-month-old mice (n=5 mice per group). FIG. 1V is a graph of memory recall in “Context A” (see FIG. 1T) 1 day after training (Test 1, n=9 mice per group). FIG. 1W provides graphs showing freezing by blue light stimulation (left panel) and average freezing for two light-off and light-on epochs (right panel). FIG. 1X is a graph of memory recall in “Context A” (see FIG. 1T) 3 days after training (Test 2). Statistical comparisons are performed using unpaired t-tests; *P<0.05, **P<0.01, ***P<0.001. Data are presented as mean±standard error of the mean (s.e.m.).

FIG. 2A-I shows neural correlates of amnesia in early AD mice. FIG. 2A-B provides photomicrographic images showing dendritic spines from DG engram cells of control (FIG. 2A) and AD (FIG. 2B) groups. FIG. 2C is a graph showing average spine density showing a decrease in AD mice (n=7,032 spines) compared with controls (n=9,437 spines, n=4 mice per group). FIG. 2D is a schematic diagram of an embodiment of study in which, for engram connectivity, MEC/LEC and DG cells were injected with virus cocktails. FIG. 2E is an engram connectivity behavioral schedule. Mice (n=4 per group) were either given a natural exploration session (−) or a PP engram terminal stimulation session (+) in an open field. FIG. 2F is a photomicrographic image showing simultaneous labelling of engram terminals (upper row) and engram cells (lower row). The labeled terminals reflect mossy cell axons. FIGS. 2G and H provide photomicrographic images showing c-Fos⁺/eYFP⁺ overlap in the DG.

FIG. 21 is a graph showing c-Fos⁺/eYFP⁺ counts from control and AD mice. Chance overlap (0.24) was calculated and indicated by the dashed line. Statistical comparisons are performed using unpaired t-tests; **P<0.01, ***P<0.001. Data are presented as mean±s.e.m.

FIG. 3A-P shows that reversal of engram-specific spine deficits rescues memory in early AD mice. FIG. 3A is a schematic diagram of an embodiment of engram-specific optical LTP using two viruses. FIG. 3B is a schematic diagram showing a virus cocktail injected into MEC/LEC. FIG. 3C-E provides photomicrographic images showing oChIEF labelling 24 h after CFC: in MEC on DOX (left; FIG. 3C) and off DOX (right; FIG. 3C); in LEC off DOX (FIG. 3D); in DG off DOX (sagittal; FIG. 3E). Scale bar shown in FIG. 3C applies to FIG. 3D-E, FIG. 3F is a graph showing oChIEF cell counts (n=3 mice per group). FIG. 3G is a schematic diagram and a graph of results in an embodiment of in vivo spiking of DG neurons in response to 100 Hz light applied to PP terminals. FIG. 3H shows an embodiment of optical LTP protocol [Nabavi, S. et al. (2014) 511, 348-352]. FIG. 3I-J provide micrographic image and graph showing in vitro responses of DG cells after optical LTP. Image showing biocytin-filled DG cell receiving oChIEF⁺ PP terminals (coronal; FIG. 3I). Normalized (Norm.) excitatory post-synaptic potentials (EPSPs) showing a 10% increase in amplitude (n=6 cells; FIG. 3J and see Examples, Methods section). FIG. 3K is a schematic diagram showing an embodiment for in vivo optical LTP at EC-DG synapses, MEC/LEC and DG cells were injected with virus cocktails. FIG. 3L shows a protocol for in vivo spine restoration of DG engram cells in AD mice (left panel); photomicrographic images showing dendritic spines of DG engram cells after LTP (middle panel). A two-way analysis of variance (ANOVA) followed by Bonferroni post-hoc tests revealed a spine density restoration in AD+100 Hz mice (f_{1,211}=7.21, P<0.01, 13,025 spines, n=4 mice per group; right panel). Dashed line represents control mice spine density (1.21). FIG. 3M provides a behavioral schedule for memory rescue in AD mice (left panel) and a graph (right panel) of results from a two-way ANOVA with repeated measures followed by Bonferroni post-hoc tests revealed restored freezing in AD+100 Hz mice (f_{1,36}=4.95, P<0.05, n=10 mice per group; right panel). Dashed line represents control mice freezing (48.53). FIG. 3N is a graph of c-Fos⁺/eYFP overlap cell counts in mice that were perfused after rescue. Chance was estimated at 0.22. NS: not significant. FIG. 3O provides results from embodiment of construct for ablation of engram cells using DTR (schematic diagram in left panel); photomicrographic images showing DG engram cells after saline/DT administration (middle panel); and a graph of DTR-eYFP cell counts (n=5 mice per group; right panel). FIG. 3P provides an embodiment of a behavioral schedule testing the necessity of engram cells after spine restoration (left panel); and a graph showing that memory recall showed less freezing of AD mice treated with DT (AD rescue+DTR+DT) compared with saline-treated mice (n=9 mice per group; right panel). Dashed line represents freezing of non-stimulated early AD mice (20.48). Unless specified, statistical comparisons are performed using unpaired t tests; *P<0.05, **P<0.01, ***P<0.001. Data are presented as mean±s.e.m.

FIG. 4A-C shows recovery of multiple types of HPC-dependent memories from amnesia in early AD. FIG. 4A provides a schematic diagram of MEC/LEC and DG cells injection with virus cocktails (left panel); and a behavioral schedule for engram labelling (right panel). FIG. 4B provides graphs showing: inhibitory avoidance (IA rescue) long-term rescue (n=10 mice per group); Recall test 1, which showed decreased latency and time on platform for AD mice. A two-way ANOVA with repeated measures followed by Bonferroni post-hoc tests revealed a recovery of IA memory in early AD mice (latency: F_{1,27}=25.22, P<0.001; time on platform: F_{1,27}=6.46, P<0.05; recall test 2 graphs). FIG. 4C provide images of heat maps showing novel object location (NOL) long-term rescue (n=15 mice per group). Average heat maps showing exploration time for familiar (F) or novel (N) locations (left or right, respectively). White circles represent object location. FIG. 4C also provides graphs of results of Recall test 1, which showed comparable exploration off familiar locations by control and AD mice; however, AD mice showed decreased exploration of novel locations. A two-way ANOVA with repeated measures followed by Bonferroni post-hoc tests revealed a recovery of NOL memory in early AD mice (F_{1,56}=5.87, P<0.05; Recall test 2 graph). Unless specified, statistical comparisons are performed using unpaired t-tests; *P<0.05, **P<0.01. Data are presented as mean±s.e.m; NS: not significant.

FIG. 5A-P shows characterization of 7-month-old early AD mice. FIG. 5A-D provides photomicrographic images showing hippocampal Aβ⁺ plaques lacking in control mice (FIG. 5A, FIG. 5B) and 7-month-old AD mice (FIG. 5C), which showed an age-dependent increase in 9-month-old AD mice (FIG. 5D). FIG. 5E-F provides photomicrographic images showing neuronal nuclei (NeuN) staining of DG granule cells in control (FIG. 5E) and 7-month-old AD (FIG. 5F) mice. FIG. 5G is a graph showing NeuN⁺ fluorescence intensity of the granule cell layer from control and AD sections shown in FIG. 5E-F (n=8 mice per group). FIG. 5H-I provides heat maps showing exploratory behavior in an open field arena from control (FIG. 5H) and 7-month old AD (FIG. 5I) mice. FIG. 5J-K provides graphs of distance travelled (FIG. 5J) and velocity (FIG. 5K), which did not differ between control and AD groups (n=9 mice per group). FIG. 5L-M are photomicrographic images showing adult newborn neurons (DCX⁺) in DG sections from control mice (FIG. 5L) that are double positive for NeuN (FIG. 5M). FIG. 5N is a graph of percentage of NeuN⁺ cells among DCX⁺ cells (n=3 mice). FIG. 5O-P provide photomicrographic images showing DCX⁺ neurons in DG sections from control (FIG. 5O) and AD (FIG. 5P) groups (n=4 mice per group). FIG. 5Q provides a graph of DCX⁺ cell counts from control and AD mice. Data are presented as mean±s.e.m.

FIG. 6A-F shows labelling and engram activation of early AD mice on DOX. FIG. 6A is a graph of home cage labeling of mice are taken off DOX for 24 h in the home cage (HC) and subsequently trained in CFC. DG sections (n=3 mice per group) revealed 2.05% ChR2-eYFP labelling in the home cage, consistent with the previously established engram tagging strategy [Liu, X. et al. (2012) Nature 484, 381-385]. FIG. 6B is a photomicrographic image of hippocampus from mice injected with a virus cocktail of AAV₉-c-Fos-tTA and AAV₉-TRE-ChR2-eYFP. After 1 day off DOX, kainic acid was used to induce seizures. Image shows efficient labelling throughout the DG. FIG. 6C is a graph of ChR2-eYFP cell counts from DG sections shown in FIG. 6B (n=3 mice). FIG. 6D provides a behavioral schedule for optogenetic activation of DG engram cells. FIG. 6E is a graph showing that memory recall 1 day after training (Test 1) showed less freezing of AD mice compared with control mice (n=8 mice per group). FIG. 6F are graphs of engram activation with blue light stimulation (left panel) and average freezing for the two light-off and light-on epochs (right panel). Statistical comparisons are performed using unpaired t-tests; **P<0.01. Data are presented as mean±s.e.m.

FIG. 7A-C shows that chronic DG engram activation in early AD mice did not rescue long-term memory. FIG. 7A is a behavioral schedule for repeated DG engram activation experiment. Ctx is “Context”. FIG. 7B is a graph of results from AD mice in which a DG memory engram was reactivated twice a day for 2 days (AD+ChR2) that showed increased STM freezing levels compared with memory recall before engram reactivation (ChR2-STM test, n=9 mice per group). FIG. 7C is a graph showing memory recall 1 day after repeated DG engram activations (ChR2-LTM test). NS: not significant. Statistical comparisons are performed using unpaired t-tests; *P<0.05, **P<0.01. Data are presented as mean±s.e.m.

FIG. 8A-M shows that engram activation restored fear memory in triple-transgenic and PS1/APP/tau models of early AD. FIG. 8A is a schematic diagram of a triple-transgenic mouse line obtained by mating c-Fos-tTA transgenic mice [Liu, X. et al. (2012) 484, 381-385][Reijmers, L. G. et al. (2007) 317, 1230-1233] with double-transgenic APP/PSl AD mice [Jankowsky, J. L. et al (2004) Hum. Mol. Genet. 13, 159-170]. These mice combined with a DOX sensitive AAV virus permits memory engram labelling in early AD. FIG. 8B is a schematic diagram illustrating triple transgenic mice injection with AAV₉.TRE-ChR2-eYFP and implanted with an optic fiber targeting the DG. FIG. 8C is a photomicrographic image showing DG engram cells of triple-transgenic mice 24 h after CFC. FIG. 8D is a graph of ChR2-eYFP cell counts from control and triple-transgenic AD mice (n=5 mice per group). FIG. 8E is a schematic diagram showing a behavioral schedule for engram activation and illustrating mouse conditions. FIG. 8F is a graph of memory recall 1 day after training (Test 1) that showed less freezing of triple-transgenic AD mice compared with control mice (n=10 mice per group). FIG. 8G are graphs of engram activation with blue light stimulation (left panel) and average freezing for the two light-off and light-on epochs (right panel). FIG. 8H is a schematic diagram showing triple-transgenic AD model (3×Tg-AD) as previously reported [Oddo, S. et al. (2003) 39, 409-421]. A cocktail of AAV₉-c-Fos-tTA and AAV₉-TRE-ChR2-eYFP viruses were used to label memory engrams in 3×Tg-AD mice. FIG. 8I is a photomicrographic image showing memory engram cells in the DG of 3×Tg-AD mice 24 h after CFC. FIG. 8J is a graph of ChR2-eYFP cell counts from DG sections of control and 3×Tg-AD mice (n=4 mice per group). FIG. 8K provides a behavioral schedule for engram activation and illustrating mouse conditions. FIG. 8L is a graph of memory recall 1 day after training (Test 1) that showed less freezing of 3×Tg-AD mice compared with control mice (n=9 mice per group). FIG. 8M provides graphs of engram activation with blue light stimulation (left panel) and average freezing for the two light-off and light-on epochs (right panel). Statistical comparisons are performed using unpaired t-tests: *P<0.05, **P<0.01. Data are presented as mean±s.e.m.

FIG. 9A-B provides data on dendritic spines of engram cells in 7-month-old early AD mice. FIG. 9A is a graph of average dendritic spine density of DG engram cells showed an age-dependent decrease in 7-month old APP/PS1 AD mice (n=7,032 spines) as compared to 5-month-old AD mice (n=4,577 spines, n=4 mice per group). Dashed line represents spine density of control mice (1.21). FIG. 9B provides graphs of spine density, with left panel showing average dendritic spine density of CA3 engram cells in control (n=5,123 spines) and AD mice (n=6,019 spines, n=3 mice per group) and right panel showing average dendritic spine density of CA1 engram cells in control (n=9,120 spines) and AD mice (n=7,988 spines, n=5 mice per group). NS: not significant. Statistical comparisons are performed using unpaired t-tests; **P<0.01 Data are presented as mean±s.e.m.

FIG. 10A-D shows the high-fidelity responses of oChIEF⁺ cells and dendritic spines of DG engram cells after in vitro optical LTP. FIG. 10A provides a schematic diagram of embodiment in which EC cells were injected with a virus cocktail containing AAV₉.TRE-oChIEF-tdTomato for activity-dependent labelling. FIG. 10B is a photomicrographic image showing a biocytin-filled oChIEF⁺ stellate cell in the EC. FIG. 10C provides traces of 100 Hz (2-ms pulse width) stimulation of an oChIEF⁺ cell across 20 consecutive trials. Spiking responses exhibit high fidelity. FIG. 10D is a graph showing that average dendritic spine density of biocytin-filled DG cells showed an increase after optical LTP induction in vitro (n=1,452 spines, n=6 cells). Statistical comparisons are performed using unpaired t-tests; *P<0.05. Data are presented as mean±s.e.m.

FIG. 11A-B shows that behavioral rescue and spine restoration by optical LTP is protein-synthesis dependent. FIG. 11A provides a modified behavioral schedule for long term rescue of memory recall in AD mice in the presence of saline or anisomycin (left panel) and a graph showing that memory recall 2 days after LTP induction followed by drug administration showed less freezing of AD mice treated with anisomycin (AD+100 Hz+Aniso) compared with saline-treated AD mice (AD+100 Hz+saline, n=9 mice per group; right panel). Dashed line represents freezing level of control mice (48.53). Ctx is “Context”. FIG. 11B is a graph showing that average dendritic spine density in early AD mice treated with anisomycin after LTP induction (n=4,810 spines) was decreased compared with saline-treated AD mice (n=6,242 spines, n=4 mice per group). Dashed line represents spine density of control mice (1.121). Statistical comparisons are performed using unpaired t-tests; *P<0.05. Data are presented as mean±s.e.m.

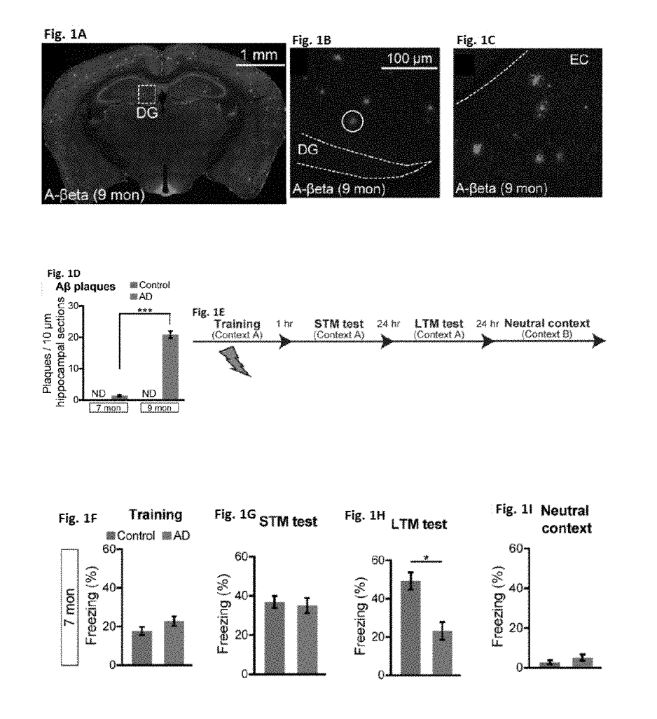

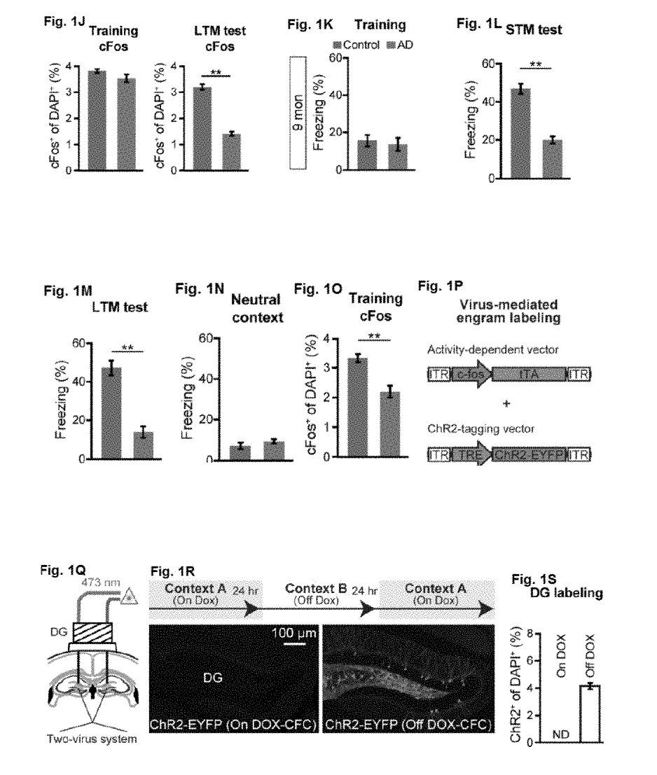

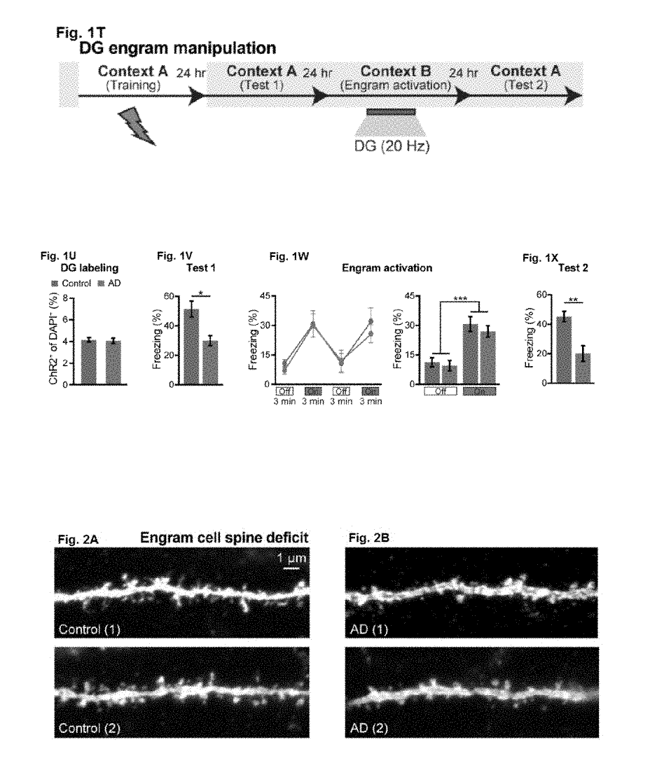

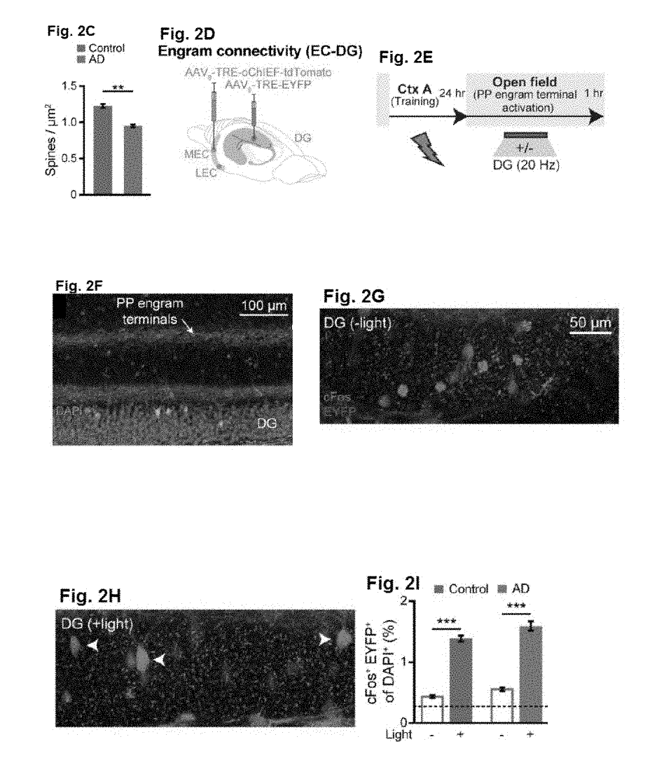

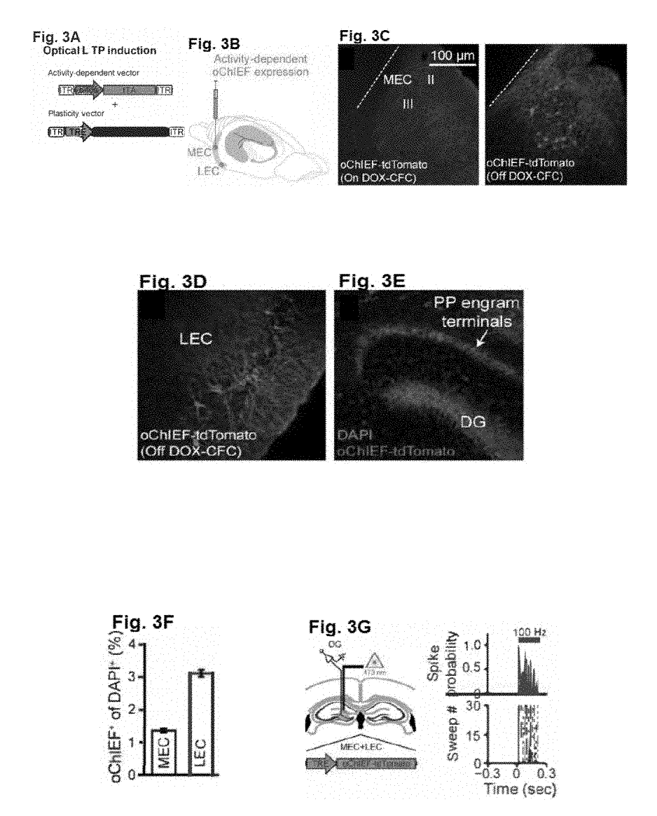

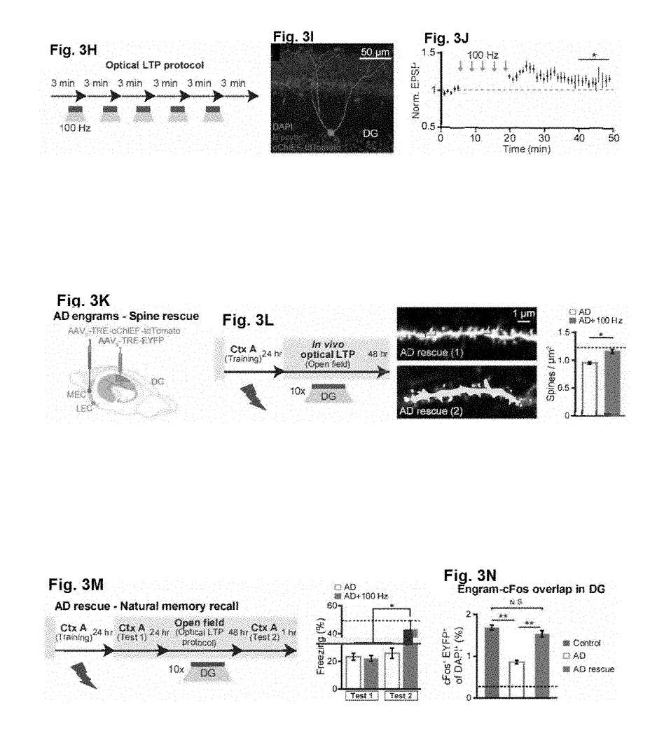

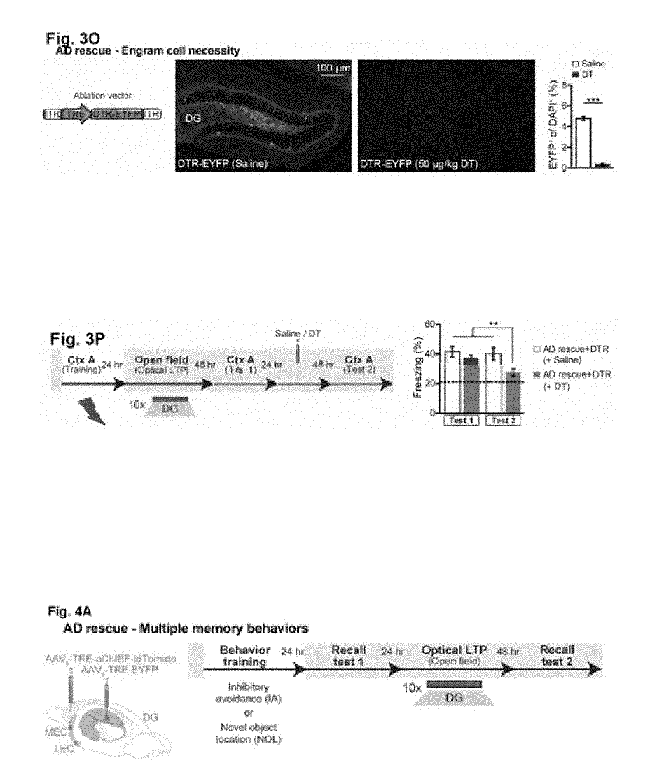

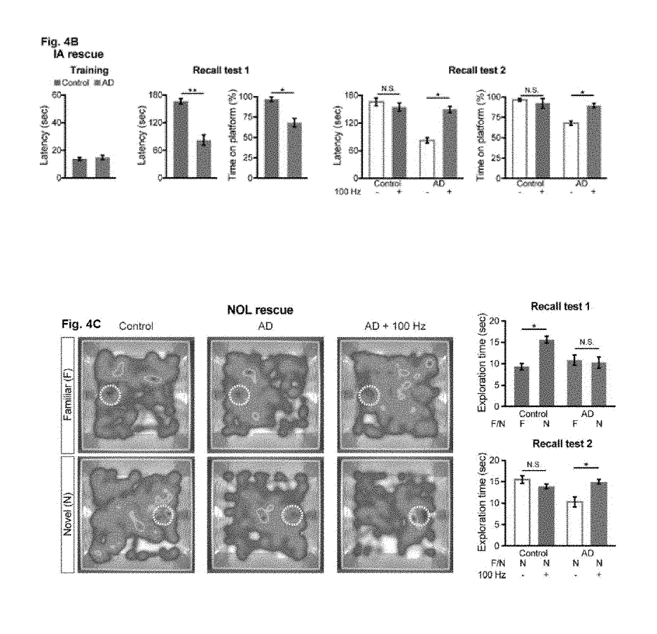

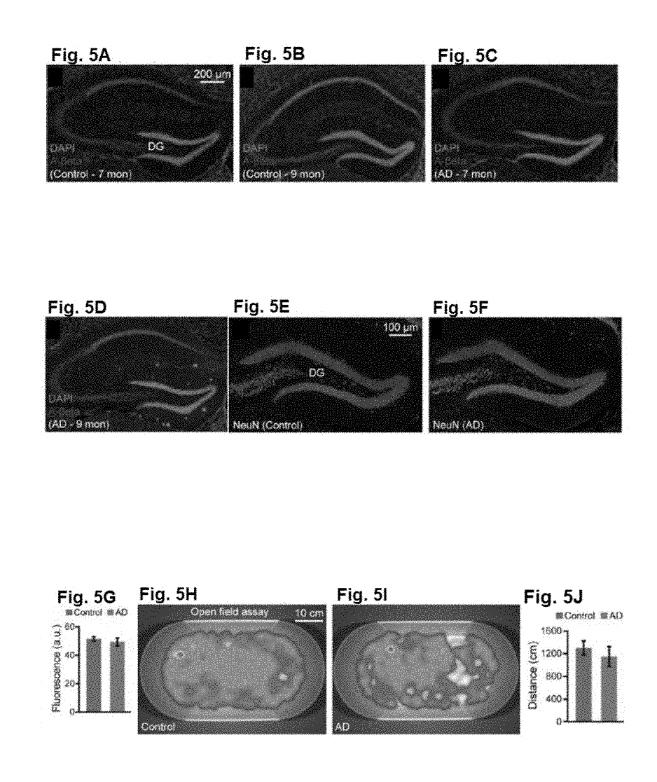

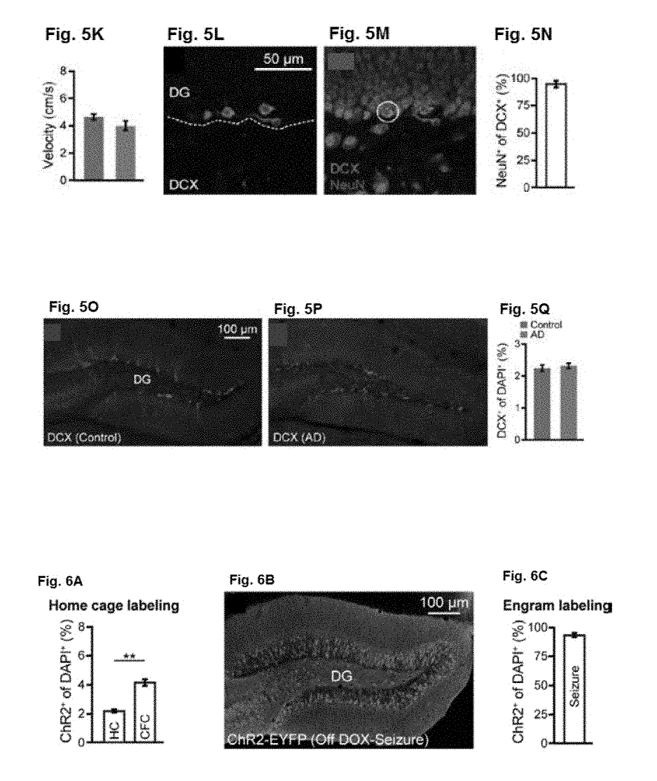

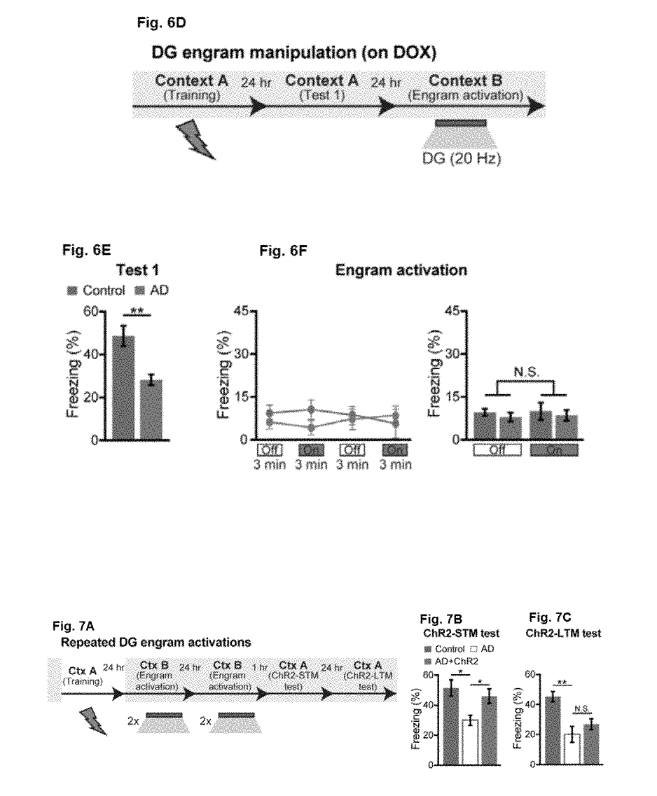

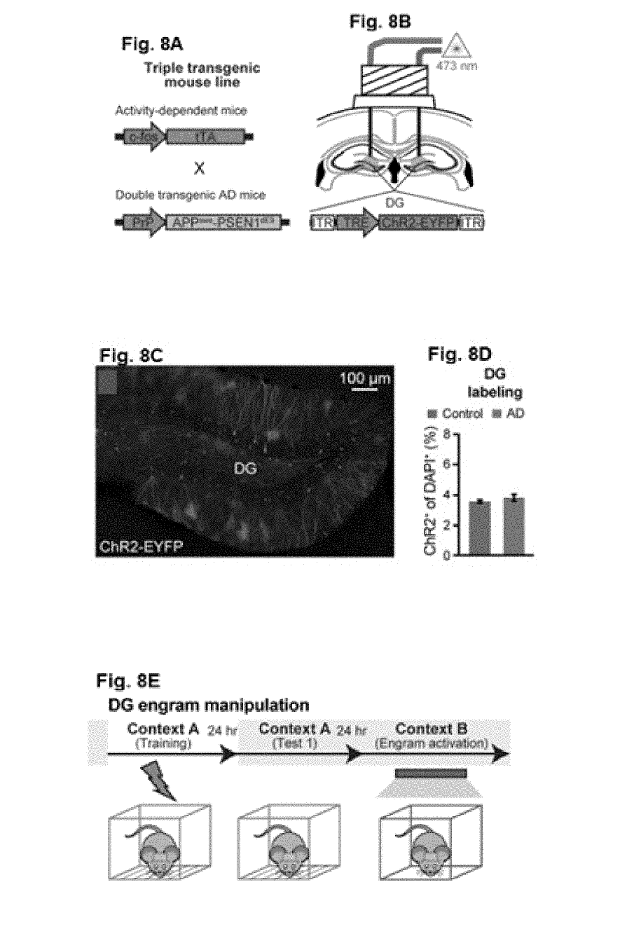

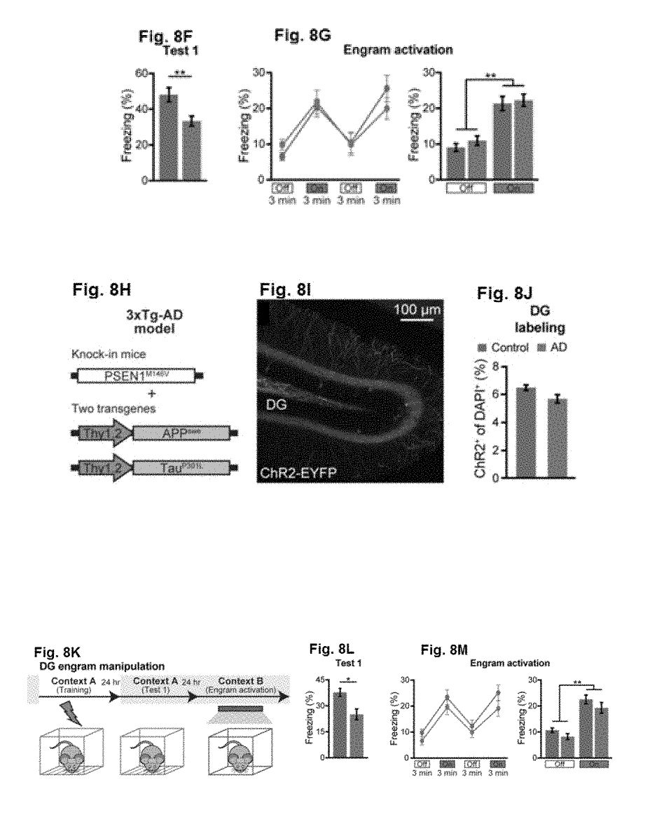

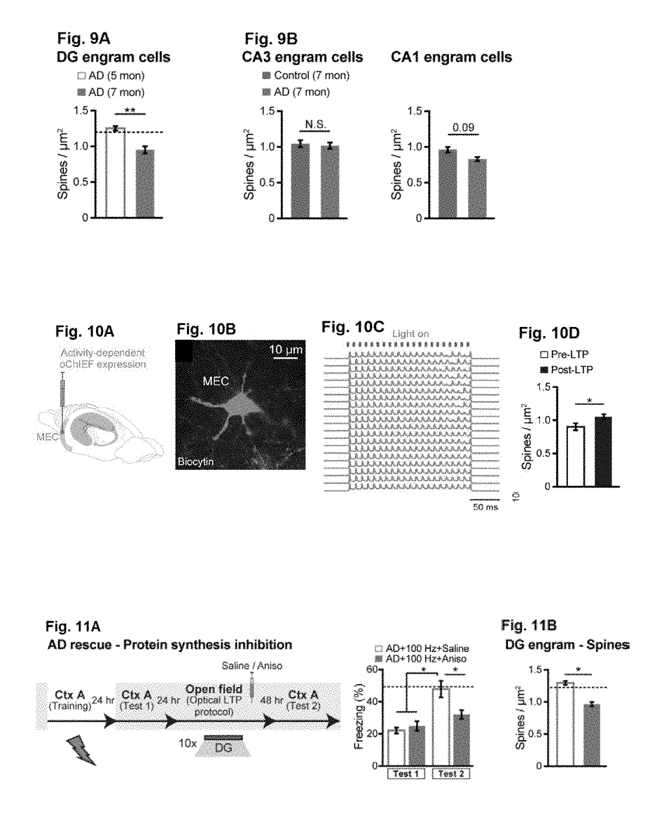

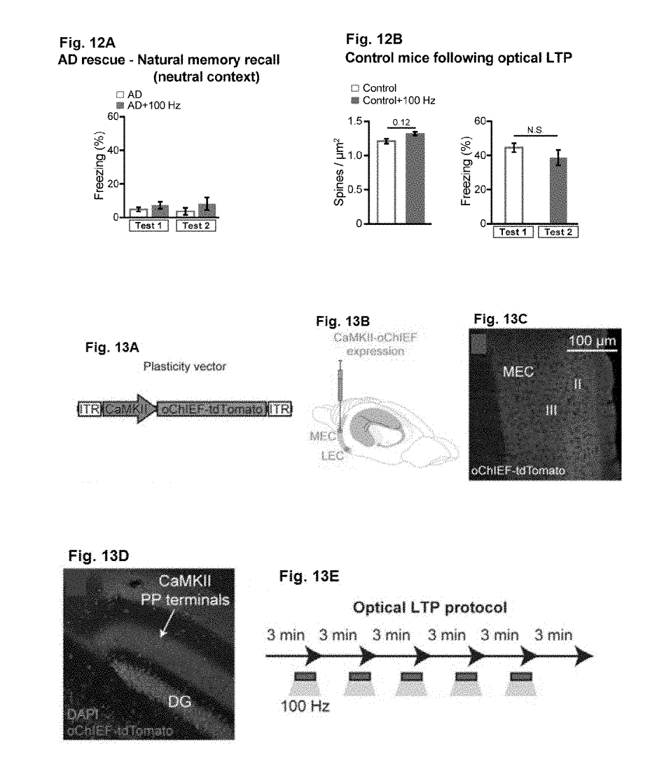

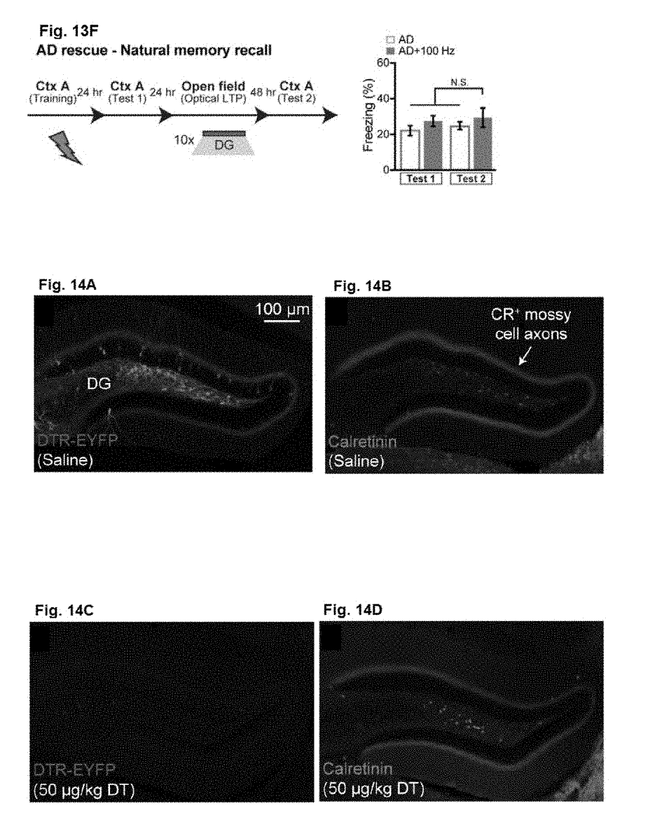

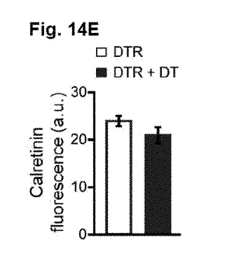

FIG. 12A-B shows rescued early AD mouse behavior in a neutral context and control mouse behavior after in vivo optical LTP. FIG. 12A is a graph of freezing after the long-term rescue of memory recall in AD mice (Test 2; from FIG. 3M), animals were placed in an untrained neutral context to measure generalization (n=10 mice per group). Rescued AD mice (AD+100 Hz) did not display freezing behavior. FIG. 12B are graphs showing in left panel, average dendritic spine density of DG engram cells from control mice remained unchanged after optical LTP induction in vivo (control+100 Hz, n=4,211 spines, n=3 mice; control data from FIG. 2C) and in right panel, the behavioral rescue protocol applied to early AD mice (from FIG. 1M) was tested in age-matched control mice (n=9 mice per group). Similar freezing levels were observed after optical LTP (Test 2) as compared to memory recall before the 100 Hz protocol (Test 1). NS: not significant. Statistical comparisons are performed using unpaired t-tests. Data are presented as mean±s.e.m.

FIG. 13A-F shows that optical LTP using a CaMKII-oChIEF virus did not rescue memory in early AD mice. FIG. 13A is a schematic diagram of an embodiment of an AAV virus expressing oChIEF-tdTomato under a CaMKII promoter. FIG. 13B is a schematic diagram showing CaMKII-oChIEF virus injected into MEC and LEC. FIG. 13C-D shows tdTomato labelling in a large portion of excitatory MEC neurons (FIG. 13C) as well as the PP terminals in the DG (FIG. 13D). FIG. 13E shows embodiment of an in vivo optical LTP protocol [Nabavi, S. et al. (2014) Nature 511, 348-352]. FIG. 13F shows embodiment of a behavioral schedule for long-term rescue of memory recall m AD mice (left panel). In contrast to the engram specific strategy, long-term memory could not be rescued by stimulating a large portion of excitatory PP terminals in the DG (FIG. 13F, right panel; n=9 mice per group). NS: not significant. Statistical comparisons are performed using unpaired t-tests. Data are presented as mean±s.e.m.

FIG. 14A-E shows normal DG mossy cell density after engram cell ablation. FIG. 14A-D provides photomicrographic images showing DG engram cells after saline treatment (FIG. 14A) and the corresponding calretinin positive (CR⁺) mossy cell axons (FIG. 14B). DTR-eYFP engram cell labelling after DT treatment is shown in FIG. 14C and the respective CR⁺ mossy cell axons are shown in FIG. 14D. FIG. 14E is a graph of CR⁺ fluorescence intensity of mossy cell axons from saline- and DT-treated DG sections that are shown in FIG. 14A-D (n=8 mice per group). Data are presented as mean±s.e.m.

### DETAILED DESCRIPTION

It has now been shown that optogenetic induction of long-term potentiation at perforant path synapses of dentate gyrus engram cells restores both spine density and long term memory. It has also now been demonstrated that an ablation of dentate gyrus (DG) engram cells containing restored spine density prevents the rescue of long-term memory. Methods of the invention, in part, include at least one of: increasing the number of dendritic spines on one or a plurality of DG memory engram cell in a subject and stabilizing the number of dendritic spines on one or a plurality of DG memory engram cells in a subject. Selective rescue of spine density in engram cells can be an effective strategy for treating memory loss in memory-impairment-associated diseases and conditions, and also, in some embodiment of the invention, can be used in methods to increase memory in a subject not exhibiting a memory-impairment-associated disease or condition. The term “engram” is a term used in the art in reference to a means by which memories are stored. Formation of engrams may include activation of neurons during the process of acquiring a memory, and resulting lasting physical or chemical changes. An engram may include encoding in neural tissue that provides a physical basis for the persistence of memory. Certain aspects of the invention include increasing the density of dendritic spines on engram cells in methods for treating memory-impairment-associated diseases and conditions.

Methods have now been identified that can be used to selectively induce high-frequency neuronal firing under conditions that produce a temporally sustained increase and/or temporally sustained stabilization in the dendritic spine density on one or a plurality of DG memory engram cells in a subject, which results in at least one of: increased memory, reduction of memory loss, reduction of memory impairment, slowing of memory loss, stabilization of memory level, halting of memory loss, and recovery from memory loss in a subject. As used herein, the term “selectively” when used in reference to inducing high-frequency neuronal firing means that the neurons in which the high-frequency firing is induced are members of a limited population of cells. For, example, unlike prior stimulation methods such as deep-brain stimulation and certain pharmacological means with which widespread stimulation occurred in brain regions, methods of the invention include stimulating a predetermined subset of memory engram cells in a specific region of a subject's brain. For example, some aspects of the invention include selectively inducing high-frequency neuronal firing by expressing a stimulus-activated opsin polypeptide in one or a plurality of EC or DG cells in a subject, wherein activating the expressed stimulus-activated opsin polypeptide selectively induces high-frequency neuronal firing in one or a plurality of DG memory engram cells in the subject.

Certain embodiments of treatment methods of the invention comprise selectively inducing high-frequency neuronal firing in one or more DG memory engram cells in a subject, wherein the high-frequency neuronal firing is induced at an amount (also referred to herein as a “level”) that is effective to increase and/or stabilize dendritic spine density in the DG memory engram cells in which the high-frequency firing was induced. It has now been determined that use of methods of the invention for suitable induction of the high-frequency neuronal firing in one or more DG memory engram cells results in a temporally sustained increase and/or stabilization in dendritic spine density on one or a plurality of the DG cells. In some aspects of methods of the invention, the described induction of high-frequency neuronal firing can be used to sustain the number of dendritic spines and/or increase the number of dendritic spines, thereby increasing the density of dendritic spines on memory engram neurons than would be present in the absence of a treatment of the invention.

Treatment methods of invention can be used to increase the density of dendritic spines on one or a plurality of DG memory engram cells in a treated subject to a density level that is higher than would be present in the subject absent the treatment of the subject with a method of the invention. Methods of the invention can be used to induce high-frequency neuronal firing in a neuronal cell that result in one or more of: (1) generating one or a plurality of new dendritic spines on the cell and (2) maintaining one or a plurality of preexisting dendritic spines on the cell. Instances in which one or a plurality of new dendritic spines are generated on one or a plurality of cells in a subject, the term “temporally sustained” means the length of time the new dendritic spine is present on the cell following its generation. Instances in which one or a plurality of existing dendritic spines are maintained on one or a plurality of cells in a subject, the term “temporally sustained” means the length of time the existing dendritic spine is present on the cell following application of the high-frequency firing to the cell. As used herein, the term “temporally sustained” may be a time period of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 days.

The ability to controllably induce temporally sustained dendritic firing in a manner that results in generation of new dendritic spines and/or maintenance of existing dendritic spines in memory engram cells resulting in one or more of: memory improvement, memory retrieval improvement, slowing of memory worsening, stopping of memory worsening, etc. with light has been demonstrated, and expression and suitable activation of selected opsins in cells in a subject can be included in methods of the invention. The present invention enables targeted expression and localization of opsins in cells that are involved in memory engrams such as DG cells, and activity of DG cells can be modulated with methods of the invention such that the cells experience high-frequency firing and a temporally sustained increase in dendritic spine density in treatments for memory-impairment-associated diseases and conditions. Treatment methods of the invention and their use have broad-ranging applications for treatment of memory loss, and to enhance memory, and in research applications, some of which are describe herein.

Different types of stimulus-activated opsin molecules (polypeptides and/or encoding polynucleotides) are known in the art and may be suitable and selected for use in embodiments of the invention. Examples of stimulus-activated opsins that may be expressed in a subject as part of a treatment method of the invention, are channelrhodopsin, halorhodopsin, Archaerhodopsin, and Leptosphaeria rhodopsin polypeptides and their encoding polynucleotides, wherein when activated under suitable conditions, the activated opsins induce high-frequency neuronal firing in one or a plurality of: the cell in which the stimulus-activated opsin is expressed, one or more cells directly downstream from the cell in which the stimulus-activated opsin is expressed, and one or more cells indirectly downstream from the cell in which the stimulus-activated opsin is expressed, wherein the frequency of the high-frequency neuronal firing is at least 45 Hz, 50 Hz, 60 Hz, 70 Hz, 80 Hz, 90 Hz, 100 Hz, or 110 Hz. Stimulus-activated opsin molecules that are able to induce high-frequency neuronal firing at a frequency of at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz in neuronal cells, including each integer within the stated range, are known in the art and have been used to alter membrane potential in electrically excitable cells. Non-limiting examples of stimulus-activated opsins that may be selected and can be used in methods of the invention are ChrimsonR, Chronos, ChEF, oChiEF, or ChIEF polypeptides or functional variants thereof, wherein the functional variant retains the ability to induce high-frequency neuronal firing that has a frequency of at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz in neuronal cells, including each integer within the stated range. Certain stimulus-activated opsins that are not suitable for use in methods of the invention are those that do not induce the high-frequency neuronal firing when used in methods of the invention. In some embodiments of the invention, a stimulus-activated opsin is not an opsin that when activated results in a frequency of neuronal firing that is less than 45 Hz, less than 40 Hz or lower. For example, though not intended to be limiting, in some embodiments of the invention, the stimulus-activated opsin is not a ChR2 opsin molecule.

Stimulus-activated opsin molecules are routinely expressed in fusion proteins and used in optogenetic methods and compositions. Expression of such an opsin in a cell permits modulation of the cell's membrane potential when the cell is contacted with a suitable stimulus, which in some instances is illumination or light. Methods to prepare and express a light-activated opsin in a cell, and in a subject, are well known in the art, as are methods to select and apply a suitable wavelength of light to the cell in which the opsin is expressed in order to activate the expressed opsin ion channel or ion pump in the cell. Methods of adjusting illumination variables and conditions for activation are well-known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8. Epub 2007 May 1; the content of each of which is incorporated herein by reference in its entirety. It will be understood that an opsin polypeptide that is activated by light or that is activated by another stimulation means can be used in aspects of compositions and methods of the invention.

The present invention, in some aspects, includes preparing polynucleotide sequences and expressing in cells and membranes polypeptides encoded by the prepared polynucleotides. In certain implementations, the invention comprises methods for preparing and using genes encoding stimulus-activated opsins such as light-activated ion channel polypeptides and light-activated ion pumps in vectors that may also include one or more additional polynucleotide molecules that encode trafficking polypeptides, detectable labels, or other molecules of interest to be expressed in a cell with the opsin polypeptide. Some embodiments of the invention include expression in cells, tissues, and subjects of one or more opsin polypeptides.

As used herein, the terms “opsin polypeptide” and “opsin amino acid sequence” when used in reference to an opsin molecule that is included in a method of the invention, means an opsin polypeptide or a functional variant thereof, and an amino acid sequence of an opsin, or functional variant thereof. Similarly, the terms “opsin polynucleotide” and “opsin nucleic acid sequence” when used in reference to an opsin molecule that is included in a vector or method of the invention, means an opsin polynucleotide or a functional variant thereof, and a nucleic acid sequence encoding an opsin or a functional variant thereof. Certain embodiments of compositions, compounds, and methods of the invention may additionally include a vector or construct that comprises such polynucleotide sequences. A stimulus-activated opsin or a functional variant of a stimulus-activated opsin that is used in a method of the invention will, when expressed in a cell and stimulated under suitable conditions, induce high-frequency neuronal firing in the cell and/or a cell directly or indirectly downstream from the cell, wherein the firing frequency will be at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz including each integer within the stated range.

### Sequences and Functional Variants

The term “variant” as used herein in the context of polypeptide molecules and/or polynucleotide molecules, describes a molecule with one or more of the following characteristics: (1) the variant differs in sequence from the molecule of which it is a variant, (2) the variant is a fragment of the molecule of which it is a variant and the fragment is identical in sequence to a portion of the sequence of which it is a variant, and/or (3) the variant is a fragment and differs in sequence from the portion of the molecule of which it is a variant. As used herein, the term “parent” in reference to a sequence means a sequence on which a variant sequence is based or originates. For example, though not intended to be limiting, a ChIEF sequence is considered to be the parent sequence for a ChIEF functional variant.

An opsin molecule that is a functional variant of a wild-type or other opsin molecule may have all or part of the sequence of its parent molecule, but with a change or modification of 1, 2, 3, 4, 5, 6, 7, 8, 9, 10, 11, 12, 13, 14, 15, 16, 17, 18, 18, 20, 21, 22, 23, 24, 25, or more amino acids or nucleic acids, compared to its parent amino acid or nucleic acid sequence, respectively. In certain aspects of the invention, an opsin molecule functional variant may have a longer sequence than its parent molecule. As used herein, a sequence change or modification in a polypeptide may be one or more of a substitution, deletion, and insertion of an amino acid, or a combination thereof. As used herein, a sequence change or modification in a polynucleotide may be one or more of a substitution, deletion, and insertion of a nucleic acid, or a combination thereof. Opsin molecules and variants thereof are known in the art and may be included in be used in methods of the invention. Standard art-known methods can be used to identify, select, and/or use an opsin polypeptide or a functional variant thereof and its encoding nucleic acid sequence in methods of the invention.

As used herein an amino acid sequence of an opsin polypeptide variant may have 85%, 86%, 87%, 88%, 89%, 90%, 91%, 92%, 93%, 94%, 95%, 96%, 97%, 98% 99% sequence identity to its parent amino acid sequence. As used herein a nucleic acid sequence encoding an opsin polypeptide variant may have 85%, 86%, 87%, 88%, 89%, 90%, 91%, 92%, 93%, 94%, 95%, 96%, 97%, 98% 99% sequence identity to its parent nucleic acid sequence. Routine sequence alignment methods and techniques can be used to align two or more similar light-activated opsin polypeptide sequences, including but not limited to wild-type and other identified opsin polypeptide sequences, thus providing a means by which a corresponding location of a modification made in one opsin polypeptide may be identified in an opsin polypeptide sequence. Such sequence alignment means can also be performed to align and identify variants from parent polynucleotide sequences for use in methods of the invention.

It is understood in the art that the codon systems in different organisms can be slightly different, and that therefore where the expression of a given protein from a given organism is desired, the nucleic acid sequence can be modified for expression within that organism. Thus, in some embodiments, an opsin and/or fusion protein of the invention is encoded by a mammalian-codon-optimized nucleic acid sequence, which may in some embodiments be a human-codon optimized nucleic acid sequence. In certain aspects of the invention, a nucleic acid sequence used in a compound, composition, or method of the invention is a sequence that is optimized for expression in a human cell.

As used herein, the terms “protein” and “polypeptide” are used interchangeably and thus the term polypeptide may be used to refer to a full-length protein and may also be used to refer to a fragment of a full-length protein, and/or functional variants thereof. As used herein, the terms “polynucleotide” and “nucleic acid sequence” may be used interchangeably and may comprise genetic material including, but not limited to: RNA, DNA, mRNA, cDNA, etc., which may include full length sequences, functional variants, and/or fragments thereof.

### Fusion-Protein Components

Certain embodiments of the invention comprise administration of a pharmaceutical composition comprising a fusion protein comprising a stimulus-activated opsin molecule, or comprising its encoding polynucleotide. Molecules that can be expressed in a fusion protein and used in an embodiment of a treatment method of the invention may include but are not limited to: one or more stimulus-activated opsin polypeptides, detectable labels polypeptides, targeting polypeptides, and trafficking polypeptides. Non-limiting examples of detectable label polypeptides include: green fluorescent protein (GFP); enhanced green fluorescent protein (EGFP), red fluorescent protein (RFP); yellow fluorescent protein (YFP), dtTomato, mCherry, DsRed, eYFP, cyan fluorescent protein (CFP); far red fluorescent proteins, etc. Numerous fluorescent proteins and their encoding nucleic acid sequences are known in the art and routine methods can be used to include such sequences in fusion proteins and vectors, respectively, of the invention.

Additional sequences that may be included in a fusion protein of the invention are trafficking, also referred to as “export” sequences, including, but not limited to: Kir2.1 sequences and functional variants thereof, KGC sequences, ER2 sequences, etc. Additional trafficking polypeptides and their encoding nucleic acid sequences are known in the art and routine methods can be used to include and use such sequences in fusion proteins and vectors, respectively, of the invention.

Stimulus-activated opsin molecules that may be included in compositions and used in certain embodiments of treatment methods of the invention includes stimulus-activated opsin molecules that when expressed in a cell and contacted with a suitable stimulus, function as a membrane channel, an ion pump, or other identified structure, based on its sequence. A stimulus-activated opsin may be an “excitatory” or “activating” when activated. A non-limiting example of an excitatory stimulus-activated opsin is an ion-channel opsin that when activated results in depolarization of the cell in which it is expressed and may induce high-frequency neuronal firing in one or more of the cell and one or more cells directly or indirectly downstream from the cell in which the opsin is expressed.

A non-limiting example of an opsin useful in certain methods of the invention is a light-activated opsin. As used herein the term “opsin” may include any opsin having a sequence that is one or more of: a wild-type opsin sequence, a modified opsin sequence, a mutated opsin sequence, a chimeric opsin sequence, a synthetic opsin sequence, a functional fragment of an opsin sequence that may include one or more additions, deletions, substitutions, or other modifications to the sequence of the parent opsin sequence from which the fragment sequence originates, and a functional variant of an opsin sequence that may include one or more additions, deletions, substitutions, or other modifications to the sequence of the parent opsin sequence from which the variant sequence originates. As used herein the term “functional” when used in reference to a fragment or variant means that the fragment or variant retains at least a portion of a function of the parent molecule. For example, a functional variant of a light-activated ion channel polypeptide differs from its parent sequence and retains at least some of the light-activated ion channel activity of its parent, including sufficient activity that when expressed in a cell and stimulated under suitable conditions, the functional variant induces high-frequency neuronal firing in the cell and/or a cell directly or indirectly downstream from the cell, wherein the high-frequency neuronal firing is at a frequency of at least 45 Hz, 50 Hz, 60 Hz, 70 Hz, 80 Hz, 90 Hz, 100 Hz, 110 Hz, 120 Hz, 130 Hz, and 140 Hz, 150 Hz, 160 Hz, 170 Hz, 180 Hz, 190 Hz, and 200 Hz in neuronal cells, including each integer within the stated range.

Methods of preparing and using opsin molecules and functional variants thereof are well known in the art and such opsins may be used in aspects of the invention. Non-limiting examples of opsins that may be included embodiments of compositions, vectors, and used in methods of the invention are: are ChrimsonR, Chronos, ChEF, and ChIEF and functional mutants (also referred to as “functional variants”) thereof. [see Klapoetke et al. (2014) Nature Methods 11(3), 338-346; for review see: Yizhar, O. et al. (2011) Neuron Vol. 71:9-34; the content of each of which is incorporated by reference herein in its entirety.] Additional opsin polypeptides that when expressed in a cell in subject and activated under suitable conditions induce high-frequency neuronal firing in the cell and/or a cell directly or indirectly downstream from the cell, that has a frequency of at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz; and their encoding nucleic acid sequences are known in the art and routine methods can be used to include and use such sequences and functional variants thereof in fusion proteins and vectors, respectively, of the invention.

### Delivery of Polypeptides

Delivery of an opsin molecule to a cell and/or expression of an opsin polypeptide in a cell can be done using art-known delivery means. In some embodiments of the invention, the term “expressing” when used in reference to administering a selected stimulus-activated opsin to a cell, means a selected stimulus-activated opsin is present in the cell as a result of administering to a subject a vector comprising the opsin's encoding polynucleotide sequence. The fusion protein is expressed from the administered vector in a cell in the subject. In certain embodiments of the invention, the term “expressing” used in reference to administering a selected stimulus-activated opsin to a cell, means a selected stimulus-activated opsin is present in the cell as a result of administering to a subject a fusion protein comprising the selected opsin.

It is well known in the art how to express and utilize fusion proteins that comprise one or more polypeptide sequences and how to prepare encoding polynucleotides for expression. In certain embodiments of the invention, a fusion protein can be expressed in a cell as a means to deliver an opsin polypeptide, such as a stimulus-activated opsin polypeptide or functional variant thereof of the invention to a cell as part of a treatment method of the invention. A fusion protein for use in methods of the invention can be expressed in a specific cell type, tissue type, organ type, and/or region in a subject, or in vitro, for example in culture, in a slice preparation, etc. Some aspects of the invention a cell in which a fusion protein is expressed or delivered is an EC cell in a subject and in some aspects of the invention a cell in which a fusion protein is expressed or delivered is a DG cell in a subject. Methods of preparation, delivery, and use of a fusion protein and its encoding nucleic acid sequences are well known in the art. Routine methods can be used in conjunction with teaching herein to express a fusion protein comprising an opsin polypeptide in a desired cell, tissue, or region in vitro or in a subject. Methods suitable to deliver fusion proteins into cells are presented herein and various methods are described in the art, [see Klapoetke et al. (2014) Nature Methods 11(3), 338-346; for review see: Yizhar, O. et al. (2011) Neuron Vol. 71:9-34, each of which is incorporated by reference herein in its entirety].

It is one aspect of the invention to provide a light-activated opsin polypeptide of the invention that is non-toxic, or substantially non-toxic in cells in which it is expressed. In the absence of light, a light-activated opsin polypeptide of the invention does not significantly alter cell health or ongoing electrical activity in the cell in which it is expressed. In some embodiments of the invention, a light-activated opsin polypeptide of the invention is genetically introduced into a cellular membrane, and reagents and methods are provided herein for genetically targeted expression of light-activated opsin polypeptides. Genetic targeting can be used to deliver a light-activated opsin polypeptide to specific cell types, to specific cell subtypes, to specific spatial regions within an organism, and to sub-cellular regions within a cell, including, cell types such as hippocampal cells, DG cells, DC cells, etc. Routine genetic procedures can also be used to control parameters of expression, such as but not limited to: the amount of a light-activated opsin polypeptide expressed, the timing of the expression, etc.

In some embodiments of the invention a composition for genetically targeted expression of a light-activated opsin polypeptide comprises a vector comprising a gene or functional variant thereof that encodes an opsin polypeptide. As used herein, the term “vector” refers to a nucleic acid molecule capable of transporting between different genetic environments another nucleic acid to which it has been operatively linked. The term “vector” also refers to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors”. Other useful vectors, include, but are not limited to viruses such as lentiviruses, retroviruses, adenoviruses, and phages. Vectors useful in some methods of the invention can genetically insert an opsin polypeptide into dividing and non-dividing cells and can insert an opsin polypeptide into a cell that is an in vivo, in vitro, or ex vivo cell.

Vectors useful in methods of the invention may comprise additional sequences including, but not limited to, one or more signal sequences and/or promoter sequences, or a combination thereof. In certain embodiments of the invention, a vector may be a lentivirus, adenovirus, adeno-associated virus, or other vector that comprises a gene encoding an opsin polypeptide. An adeno-associated virus (AAV) such as, but not limited to, AAV8, AAV1, AAV2, AAV4, AAV5, AAV9, is a non-limiting example of a vector that can be used in some embodiments of the invention to express a fusion protein of the invention in a cell and/or subject. Expression vectors and methods of their preparation and use are well known in the art. Non-limiting examples of suitable expression vectors and methods for their use are provided herein.

Promoters that may be used in methods and vectors of the invention include, but are not limited to, cell-specific promoters or general promoters. Non-limiting examples of promoters that can be used in vectors of the invention are: ubiquitous promoters, such as, but not limited to: CMV, CAG, CBA, and EF1a promoters; and tissue-specific promoters, such as but not limited to: Synapsin, CamKIIa, GFAP, RPE, ALB, TBG, MBP, MCK, TNT, and aMHC promoters. Methods to select and use ubiquitous promoters and tissue-specific promoters are well known in the art. A non-limiting example of a tissue-specific promoter that can be used to express a light-activated opsin polypeptide in a cell such as a neuron is a synapsin promoter, which can be used to express an opsin polypeptide in embodiments of methods of the invention. Additional tissue-specific promoters and general promoters are well known in the art and, in addition to those provided herein, may be suitable for use in compositions and methods of the invention.

In certain embodiments of the invention, a promotor included in a vector for expressing a fusion protein in a dentate gyrus cell is, in some aspects of the invention, a general promotor, such as but not limited to: CaMKIIa. In other embodiments of the invention, a promotor included in a vector for expressing a fusion protein in a dentate gyrus cell is a dentate-specific promoter, a non-limiting example of which is POMC. In addition, vectors use in some embodiments of the invention comprise: immediate early gene (IEG) promoters. Use of IEG promoters permits the identification of, and tagging of, memory engram cells in a living animal. Numerous IEG promoters are known and routinely used in the art, including, but not limited to c-fos, Arc, NPAS4, c-jun, Egr-1, junB, etc. [see Kovács, K. J., (2008) J. Neuroendocrinology, 20,665-672, the content of which is incorporated by reference herein in its entirety].

Methods of the invention comprise delivery of one or more vectors encoding fusion proteins to neurons. For example, a viral vector encoding a fusion protein comprising a light-activated opsin polypeptide can be delivered into small brain regions using stereotactic injection methods. Such methods permit use of known specific brain co-ordinates to select a delivery location and the stereotactic method can be used to selectively target small brain regions, for example, approximately 0.2 mm³ to 0.5 mm³, 0.1 mm³ to 0.7 mm³, 0.1 mm³ to 1.0 mm³, or up to 5.0 mm³ regions in the brain of the subject. When delivering a vector into the dentate gyrus or entorhinal cortex in a method of the invention this method of selective delivery of a vector to one or more small physical areas in the brain can be used with high efficiency in methods of the invention. In some aspects of the invention vector delivery methods may include, MRI-guided methods, convection-enhanced delivery methods, real-time imaging methods, etc. Use of such delivery methods is known in the art, see for example: Salegio, E. A. et al., (2012) Adv Drug Deliv Rev. 2012 May 15; 64(7): 598-604 and Fiandaca, M. S., et al., (2012) Pharmaceuticals, 5:553-590, the contents of each of which is incorporated by reference herein in its entirety.

In some aspects of the invention, delivery of a viral vector to one or more cells in the brain of a subject, for example EC and/or DG cells in a human or other animal brain, can be done using a viral vector strategy described herein or with another suitable art-known delivery method. In certain embodiments of methods of the invention, a vector is delivered that comprises a selected light-activated opsin polypeptide and an immediate early gene (IEG) promoter, and the IEG selectively introduces the light-activated opsin into cells in the subject that are active during a specific time window—and these active cells are memory engram cells, which will then comprise the delivered and expressed opsin at a subsequent time. After the delivery of the vector and expression of the light-activated opsin polypeptide in cells of the subject, light can be delivered to a focused or broad brain region using parameters suitable to activate the delivered opsin and to result in induction of high-frequency neuronal firing in the one or more DG memory engram cells in the subject. Thus, because only cells (i.e., memory engram cells) that include a delivered opsin will respond to contact with light, suitable stimulatory light can be applied to a region of the subject's brain that is larger in size than the region in which the light-activated opsin is located. Thus, illumination suitable to activate one or a plurality of light-activated opsins delivered using methods of the invention, need not be restricted in its spatial application such that only cells that contain an expressed light-activated opsin polypeptide are contacted with the light, because non-memory engram cells are not affected.

### Stimulation

Certain embodiments of treatment methods of the invention include stimulating a stimulus-activated opsin that has been expressed in a cell in a subject. Stimulation of a targeted opsin with a suitable stimulation means to activate the opsin in the cell. Methods of stimulating opsin polypeptides are well known in the art and may include contacting a cell that expresses an opsin with a light under suitable conditions to activate the opsin.

Methods and apparatus for contacting an expressed opsin with a suitable wavelength of light to activate an opsin ion channel polypeptide or ion pump polypeptide are known in the art. It will be understood that a light of appropriate wavelength for activation will have a power and intensity appropriate for activation of that opsin. It is known in the art that light pulse duration, intensity, and power are some of the characteristics and parameters that can be altered when activating a light-activated ion channel or ion pump with light. One skilled in the art will be able to use routine procedures to adjust power, intensity, timing, interval of stimulation, etc. appropriately when using a wavelength suitable to activate a selected opsin expressed in a method of the invention. Using standard procedures, illumination variables can be altered or “tuned” to optimize activity of a stimulus-activated opsin polypeptide when expressed in a subject and used in a treatment method of the invention. Altering illumination variables such as, but not limited to: wavelength, intensity, pulse width, pulse duration, pulse intervals, overall illumination duration, etc. can be used in conjunction with methods of the invention to optimize treatment for a particular subject, for example to increase activity of the expressed opsin polypeptide and to induce high-frequency neuronal firing in DG memory engram cells in the subject. Methods of adjusting illumination variables are well known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8 Epub 2007 May 1, each of which is incorporated by reference herein in its entirety.

It is possible to utilize a narrow range of one or more illumination characteristics to activate a light-activated opsin polypeptide expressed in a subject in a treatment method of the invention. This may be useful to illuminate a light-activated opsin polypeptide that is co-expressed with one or more other light-activated opsins (e.g., channels, pumps, etc.) that can be illuminated with a different set of illumination parameters (for example, though not intended to be limiting, different wavelengths) for their activation, thus permitting controlled activation of a mixed population of light-activated channels and/or pumps. In certain aspects of the invention, methods of treatment include expression of one type of opsin in selected cell types in a subject, and other aspects of the invention include expression of two or more different types of opsins in selected cell types in a subject. As a non-limiting example, a method of the invention may include expressing in a plurality of EC and/or DG cells in a subject a light-activated opsin that is activated strongly by contact with only blue light and also expressing in EC and/or DG cells in the subject a light-activated ion channel or light-activated ion pump that is activated using a different wavelength of light, and that may not be activated by the blue light. The expression of more than one light-activated opsin in a subject may be used in embodiments of treatment methods of the invention that include expressing one light-activated opsin in a certain population of cells in a subject and optionally, expressing a different light-activated opsin in a separate population of cells, and performing one or more of: contacting cells with different wavelengths of light to activate, activating the different opsins at different time, activating the different opsins for different lengths of time, etc., that are appropriate to activate the opsins and either excite or inhibit activity of the two populations of cells.

In some aspects of the invention, a subject receiving stimulation of an expressed light-activated opsin as part of a treatment method of the invention, is engaged during all or a portion of the stimulation period in a learning activity. Learning activities, also referred to as training activities, are well known in the art and can be selected as appropriate for a subject undergoing treatment of the invention. Non-limiting examples of learning activities include: visual training activities, memory activities, mathematical activities, puzzles, writing activities, auditory activities, motor activities, olfactory activities, reading comprehension, etc. During a learning activity, such as but not limited to those described herein, one or more memory engram cells are tagged in the brain. One or more days after learning period in a subject, engram cell stimulation is carried out and improves and/or modulates retrieval of the memory information from the learning activity. In addition, learning activities, including but not limited to those disclosed herein can be used to assess the presence or absence of a memory-impairment associated disease or condition and to assess changes in the stage of such a disease or condition in a subject.

### Methods of Using Stimulus-Activated Opsins

Opsin polypeptides are well suited for use in methods of the invention to increase activity in one or more cells in neural pathways associated with memory and to increase the density and maintenance of dendritic spines on memory engram cells such as dentate gyrus cells. Some embodiments of the invention include expressing a light activated opsin in a cell that when activated induces high-frequency neuronal firing in the cell and/or a cell directly or indirectly downstream from the cell, wherein the high-frequency neuronal firing is at a frequency of at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz. Such methods of the invention can be used to increase memory retrieval in a subject, treating a memory-impairment-associated disease or condition, and increasing memory in a normal subject, for example a subject not diagnosed with or suspected of having a memory-impairment-associated disease or condition. In some embodiments of the invention, a fusion protein comprising a light-activated opsin polypeptide can be expressed in a cell-specific, localized manner. For example, in certain aspects of the invention, a stimulus-activated opsin polypeptide is expressed in at least one of an entorhinal cortex (EC) cell and a dentate gyrus (DG) cell.

Certain embodiments of methods of the invention include expressing a light-activated opsin polypeptide in a first cell, contacting the expressed light-activated opsin polypeptide with a light suitable to activate the first cell, wherein the resulting activation of the first cell either directly or indirectly activates at least one additional cell in a manner that results in an increase in dendritic spine density on at least one of the cells. A non-limiting example of direct activation of one cell by another is an entorhinal cortex (EC) cell that projects to a dentate gyrus (DG) cell and activation of the EC cell using a method of the invention results in high-frequency neuronal firing in the EC memory engram cell that generates and/or maintains dendritic spines on the EC cell. Thus, certain embodiments of methods of the invention can comprise activating one cell wherein that activation directly activates a second cell. In certain of such embodiments, a downstream cell does not express the opsin expressed in the upstream cell. In some aspects of the invention a downstream cell does express the opsin expressed in the upstream cell. In some aspects of the invention a downstream cell may express one or more of: the same opsin and a different opsin than the opsin expressed in the upstream cell. A non-limiting example of indirect activation of one cell by another is an EC cell that projects to a DG cell that in turn projects to another cell, wherein activation of the EC cell using a method of the invention results in high-frequency neuronal firing of the DG cell, and also activation of one or more cells downstream from the DG cell. Thus, certain embodiments of methods of the invention can comprise activating one cell wherein that activation indirectly activates a one or more additional, downstream cells.

It has now been identified that stimulating a cell in the EC of a subject using methods of the invention can result in high-frequency stimulation of a second cell that is a DG memory engram cell. As used herein, the EC cell is referred to as being “upstream” in relation to the DG cell, meaning that stimulation of the EC cell results in activation of the DG cell. A first cell may be directly or indirectly upstream in relation to a second cell. For example, an EC cell can be considered to be directly upstream of the DG cell and indirectly upstream of another cell that is downstream of the DG cell. As a non-limiting example, in the hippocampus, information is transferred from the DG to subregion CA3 to subregion CA2 to subregion CA1. Therefore, in the example of activating EC engram cells that project to DG engram cells, cells in CA3 or CA2 or CA1 can be considered to be indirectly downstream of the DG cell. Those skilled in the art will be aware of additional cells that may be directly or indirectly downstream from EC and/or DG engram cells.

It has now been identified that methods of the invention can be used to induce high-frequency firing in DG cells in a subject and the high-frequency firing increases the dendritic spine density on the DG cells and enhances memory retrieval in the subject. Thus, a method of the invention may comprise expressing a light-activated opsin in one or more cells in the EC contacting the cells with a suitable light to activate the opsin, thereby activating inducing high-frequency neuronal firing in one or more DG cells to which the activated EC cells project. Methods of the invention can be used to express one or more light-activated opsins in a specific cell type, which is then contacted with a suitable illumination to activate the opsin and activate electrical activity in that cell, resulting in high-frequency neuronal firing in a downstream cell. It will be understood that the type and level of electrical activity and ion flux in a cell expressing a light-activated opsin, will depend, in part, on the light-activated opsin that is expressed in the cell as part of the fusion protein used in methods of the invention. As described herein, a light-activated opsin suitable for use in methods of the invention is a light-activated opsin that when expressed in a cell in a subject and activated with a suitable light induces high-frequency firing in DG cells in a subject and the high-frequency firing increases the dendritic spine density on the DG cells and enhances memory retrieval in the subject.

Art-known methods can be used to select suitable stimulation parameters such as type of stimulation, illumination wavelength, intensity, illumination pulse rate, etc. for use with light-activated opsin expressed in cells and membranes in embodiments of methods of the invention. See for example: U.S. Pat. No. 8,957,028; U.S. Pat. No. 9,309,296; U.S. Pat. No. 9,284,353; U.S. Pat. No. 9,249,234; U.S. Pat. No. 9,101,690; PCT Pub. No. WO2013/07123; US Pat Pub No. 20120214188; US Pat Pub. No. 20160039902; US Pat Pub No. 20140223679; Packer, A. M. et al., 2012 Nature Methods December 9(12): 1202-1205; and Oron, D. et al., Progress in Brain Research, Chapter 7, Volume 196, 2012, Pages 119-143: the content of each of which is incorporated by references in its entirety herein.

Certain aspects of the invention include methods for modulating one or more characteristics of a cell, such as, but not limited to: electrical activity in a cell and ion flux across a cell membrane. Compositions and methods of the invention can be used in a cells in subjects as a means with which to: modulate ion flux across a membrane of a cell, treat a memory-impairment-associated disorder or condition in the subject, identify a candidate agent that when contacted with a cell expressing a fusion protein of the invention modulates electrical activity in the cell, identify an agent or event that reduces dendritic spine density in a memory engram cell that can reversed, slowed, or stopped using methods of the invention; etc. In some aspects of the invention, embodiments of methods described herein can be used to detect an effect of activating an opsin polypeptide that has been expressed in a cell and/or in a cell in a subject as part of a fusion protein of the invention. Numerous methods for expressing one or more light-activated opsin polypeptides in a host cell and/or a host subject are known in the art and the compositions and methods of the invention may be used in conjunction with such methods to enhance selective induction of high-frequency neuronal firing of memory engram cells in a subject treated with a method of the invention to produce a temporally sustained increase in dendritic spine density in the memory engram cells.

Methods of the invention permit selective expression of a light-activated ion channel polypeptide in a predetermined cell type in a subject, followed by activation of that cell using illumination. Methods and compositions of the invention provide an efficient and selective means to localize light-activated opsin polypeptides in specific cell types such as, but not limited to: entorhinal cortex cells, hippocampal cells, and dentate gyrus cells; and then to activate the expressed opsin polypeptide to modulate and control activity of the cell in which the opsin is expressed in a manner that results in high-frequency neuronal firing in cells sufficient to produce temporally sustained increase in dendritic spine density the cells in which the high-frequency neuronal firing occurred. Working operation of a prototype of this invention has been demonstrated in vivo, by genetically expressing a fusion protein comprising an opsin polypeptide in specific cells in a subject, illuminating the cells with suitable wavelengths of light to activate the opsin and to result in high-frequency neuronal firing in memory engram cells which results in a temporally sustained increase in dendritic spines in memory engram cells and increase in memory retrieval in the subject.

### Cells and Subjects

A cell used in methods of the invention may be: a cell in a subject. In some aspects of the invention, a stimulus-activated opsin polypeptide is expressed in a first cell in a subject and in some aspects of the invention, that cell is an excitable cell. A first cell that includes an expressed stimulus-activated opsin polypeptide may project to at least one additional cell and in some aspects of the invention, the at least one additional cell is an excitable cell. It will be understood that the term “excitable” refers to the ability of certain cells to be electrically excited resulting in the generation of action potentials. Examples of cells that are known as excitable cells are neurons, muscle cells (skeletal, cardiac, and smooth), and some endocrine cells (e.g., insulin-releasing pancreatic β cells). Entorhinal cortex (EC) cells and dentate gyrus (DG) cells are excitable cells. In certain aspects of the invention, one or more cells that are downstream from a cell in which a selected light-activated opsin polypeptide is expressed in a method of the invention, is an excitable cell. Examples of types of cells in which a fusion protein comprising a stimulus-activated opsin polypeptide can be delivered (expressed) in methods of the invention include but are not limited to: cells in a tissue, cell in a subject, cells in an organ, cell in a neural network, cells in a neural pathway, a cell in a brain, etc.

In certain aspects of the invention, a selected light-activated opsin polypeptide is expressed in one cell, or in a plurality of cells in a subject. As used herein, the term “plurality” of cells means two or more cells. A non-limiting example of a cell in which a selected light-activated opsin polypeptide may be expressed in a treatment method of the invention is a vertebrate cell, which in some embodiments of the invention is a mammalian cell, which in some embodiments of the invention is a human cell. Examples of cells in which a stimulus-activated opsin polypeptide can be expressed, and cells that can be activated by a cell in which a stimulus-activated opsin polypeptide is expressed include but are not limited to EC cells and DG cells. A stimulus-activated opsin may, in embodiments of the invention, be expressed in one or more of: a single cell, a cell that is one of a plurality of cells, a cell that is part of a projection or circuit of two or more directly or indirectly connected cells, and a cell that is one of two or more cells that are in physical contact with each other.

Non-limiting examples of cells that may be used in methods of the invention, or to which methods of the invention may be applied, include but are not limited to: cells that are one or more of: nervous system cells, neurons, entorhinal cortex cells, dentate gyrus cells, and hippocampal cells. In some embodiments of the invention, a cell in which a stimulus-activated opsin is expressed and activated in a manner to increase dendritic spine density as part of a method of the invention, is an EC or DG cell in a subject who is at least one of: diagnosed as having, suspected of having, or considered to be at risk of having a memory-impairment-associated disease or condition. In some embodiments of the invention, a cell in which a stimulus-activated opsin is expressed and activated in a manner to increase dendritic spine density as part of a method of the invention, is an EC or DG cell in a subject who is has not been diagnosed as having and is not suspected of having a memory-impairment-associated disease or condition. Thus, in some aspects of the invention, methods of the invention can be used to increase dendritic spine density in memory engram cells in a subject considered to have “normal” memory and memory retrieval abilities. In such subjects, methods of the invention may be used to increase dendritic spine density in memory engram cells and improve memory retrieval in the subject compared to a control pre-treatment level for the subject. As described in more detail elsewhere herein, a control level can be determined in a subject prior to a first or subsequent treatment of a subject with the method of the invention. A control level of a “normal” subject or in a subject who is at least one of: diagnosed as having, suspected of having, or considered to be at risk of having a memory-impairment-associated disease or condition may be understood to be the subject's baseline memory level and/or memory retrieval level and later memory testing can be used to assess efficacy of a method of the invention. Methods of memory testing are well known in the art and can be used to assess efficacy of methods of the invention.

In some embodiments of the invention, a cell used in conjunction with methods and compositions of the invention may be a cell in a subject at least one of: diagnosed as having, or suspected of having, and considered to be at high risk of having a memory-impairment-associated disease or condition to be treated. Non-limiting examples of cells to which treatment methods of the invention may be applied are one or more of: an EC cell in a subject and a DG cell in a subject, wherein the subject is at least one of: diagnosed as having, suspected of having, and considered to be at high risk of having a memory-impairment-associated disease or condition to be treated. In some embodiments of the invention, a cell may be a control cell. In some aspects of the invention a cell or subject can be a model cell or subject, respectively for a memory-impairment-associated disease or condition, a normal memory level, or an enhanced memory level.

In certain embodiments of the invention, a fusion protein comprising a light-activated opsin polypeptide may be expressed in one or more cells in a subject (in vivo cells). Light-activated opsin polypeptides expressed in fusion proteins may be delivered to and expressed in and activated in living subjects, etc. As used herein, the term “subject” may refer to a: human, non-human primate, cow, horse, pig, sheep, goat, dog, cat, rodent, or other host organism. As used herein the term “host” means the subject or cell in which stimulus-activated opsin polypeptide is expressed as part of an embodiment of a treatment method of the invention. In some aspects of the invention a host is a vertebrate subject. In certain embodiments of the invention, a host is a mammal. In certain aspects of the invention a host is a human.

### Controls and Candidate Compound Testing

Using certain embodiments of methods of the invention, one or more light-activated opsin polypeptides can be expressed in a localized region of subject, for example, in the EC and/or DG, and methods to stimulate and determine a response in the cell or in the subject to activation of the light-activated opsin polypeptide can be utilized to assess changes in cells, tissues, and subjects in which they are expressed. Some embodiments of the invention include directed delivery of light-activated opsins to a cell in a subject to identify effects of one or more candidate compounds on the cell, tissue, and/or subject in which the light-activated opsin is expressed. Results of testing one or more activities of a light-activated opsin polypeptide of the invention can be advantageously compared to a control. Controls may also be used to assess efficacy of a treatment method of the invention. For example, a control memory level or memory retrieval ability of a subject can be determined prior to a first, second, or subsequent treatment with a method of the invention and compared to memory level or memory retrieval ability in the subject at one or more post-treatment times. A control may also be based on results obtained from memory and memory retrieval testing in one or a plurality of individuals other than the subject.

As used herein a control may be a predetermined value, which can take a variety of forms. It can be a single cut-off value, such as a median or mean. It can be established based upon comparative groups, such as cells and subjects that include a light-activated opsin polypeptide of the invention and are contacted with insufficient light to induce high-frequency neuronal firing, subjects not treated with a method of the invention, etc. but that are tested for memory and memory retrieval under the same testing condition as a treated subject. Another example of comparative groups may be subjects having a memory-impairment-associated disorder or condition and groups without the disorder or condition. Another comparative group may be cells from a group with a family history of a memory-impairment-associated disease or condition and cells from a group without such a family history. A predetermined value can be arranged, for example, where a tested population is divided equally (or unequally) into groups based on results of testing. Those skilled in the art are able to select appropriate control groups and values for use in comparative methods of the invention.

### Methods of Treating

Some aspects of the invention include methods of treating a memory-impairment-associated disorder or condition in a subject by expressing a fusion protein comprising a selected light-activated opsin in a cell of a subject, activing the expressed opsin under suitable conditions to induce high-frequency neuronal firing in one or a plurality of memory engram cells in the subject in a manner that is sufficient to increase dendritic spine density in one or a plurality of DG cells in the subject as a treatment for the disorder or condition. Embodiments of treatment methods of the invention may include: administering to a subject in need of such treatment, a therapeutically effective amount of a vector encoding a fusion protein comprising a selected light-activated opsin polypeptide to treat the disorder or condition. Characteristics of an appropriate selected light-activated opsin polypeptide are described elsewhere herein. In certain aspects of the invention, a therapeutically effective amount of a cell that comprises a fusion protein comprising a selected light-activated opsin polypeptide may be administered to a subject in a treatment method of the invention. It will be understood that a treatment may be a prophylactic treatment or may be a treatment administered following the diagnosis and/or stage determination of a memory-impairment-associated disease or condition. A treatment of the invention may temporarily or permanently reduce or eliminate one or more symptoms or characteristics of a memory-impairment-associated disease or condition or may eliminate the memory-impairment-associated disease or condition. It will be understood that a treatment of the invention may reduce or eliminate progression of a memory-impairment-associated disease or condition and may in some instances reverse progression (e.g., result in regression) of the memory-impairment-associated disease or condition. A treatment need not entirely eliminate the memory-impairment-associated disease or condition to be effective.

In certain aspects of the invention, a means of expressing in a cell of a subject a fusion protein comprising a selected light-activated opsin polypeptide may comprise: administering to a cell in a subject a vector that encodes a fusion protein comprising the selected opsin polypeptide; administering to a subject a cell in which a fusion protein comprising the selected opsin polypeptide is present; or administering a fusion protein comprising the selected opsin polypeptide to a subject. Delivery or administration of a fusion protein for use in a method of the invention may include administration of a pharmaceutical composition that comprises a cell, wherein the cell expresses the selected opsin polypeptide. Administration of an opsin polypeptide, may, in some aspects of the invention include administration of a pharmaceutical composition comprising a vector, wherein the vector comprises a nucleic acid sequence encoding the selected opsin polypeptide, wherein the administration of the vector results in expression of a fusion protein comprising the selected opsin polypeptide in one or more cells in the subject. In some aspects of the invention, targeted expression of a selected opsin polypeptide in a particular cell type in a subject may be part of a treatment method. It will be understood that in some aspects of the invention, the starting level of expression of a particular selected opsin in a cell in a subject may be zero and a treatment method of the invention may be used to increase that level above zero. In certain aspects of the invention, for example in a subsequent delivery of a fusion protein comprising the selected opsin polypeptide to a subject, a level of expression of the opsin may be greater than zero, with one or a plurality of the selected opsin polypeptides present the subject, and a treatment method of the invention may be used to increase the expression level of the selected opsin polypeptide in the subject. As used herein, the terms: “administer” and “deliver” in the context of a treatment method of the invention include any means suitable to result in expression of a selected stimulus-activated opsin in a cell in a subject. Delivery or administration may include means such as, but not limited to: vector delivery to the subject, fusion protein delivery to the subject, cell delivery to the subject, etc.

An effective amount of a stimulus-activated opsin polypeptide in a treatment method of the invention is an amount that results in expression of the opsin in subject at a level or amount that when the opsin is activated it induces high-frequency neuronal firing in a memory engram cell such as a DG cell in the subject resulting in a temporally sustained increase in dendritic spine density on the DG cell, which is beneficial for the subject. An effective amount may also be determined by assessing physiological effects of administration on a subject such as a decrease in symptoms of a disorder or condition to be treated, following administration. For example, memory retrieval can be tested and assessed in a subject before and after the subject is treated using a method of the invention to assess whether an effective amount of one or more of: the selected opsin and illumination has been delivered to, and activated in, the subject. Other behavioral and functional assessments for memory and memory retrieval will be known to a skilled artisan and can be employed for measuring a level of a response to a treatment of the invention. The amount of a treatment may be varied for example by increasing or decreasing the amount of the selected opsin polypeptide administered, by changing the therapeutic composition in which the opsin polypeptide is administered, by changing the route of administration, by changing the dosage timing, by changing expression conditions of a fusion protein, by changing the stimulation parameters (wavelength, frequency, interval of stimulation, length of stimulation, etc.) of the expressed opsin polypeptide, and so on, each of which can assessed and implemented using routine procedures. The term: “stimulation period” when used herein in reference to a stimulation-activated opsin polypeptide, means the time beginning when the stimulus is applied to the stimulus-activated opsin until the cessation of the stimulus applied to the expressed opsin. In some embodiments of the invention, a stimulation period begins at the time the light-stimulated opsin is contact with a light suitable to activate the opsin and ends at the time the contact of the opsin with that light ceases. It will be understood that each time contact of an expressed opsin with a light for activation begins and ends—that is a stimulation period. A subject, in which a selected light-activated opsin polypeptide has been expressed as part of a method of the invention, may receive one stimulation period or may undergo a plurality of stimulation periods.

An effective amount of a selected stimulus-activated opsin polypeptide and its activation will vary with the particular condition being treated, the age and physical condition of the subject being treated; the severity of the condition, the duration of the treatment, the nature of the concurrent therapy (if any), the specific route of administration, and the like factors within the knowledge and expertise of a health practitioner. For example, an effective amount may depend upon the location and number of cells in the subject in which the opsin polypeptide is to be expressed. An effective amount may also depend on the location of the tissue to be treated. Factors useful to determine an effective amount of a therapeutic compound or composition are well known to those of ordinary skill in the art and can be addressed with no more than routine experimentation. It is generally preferred that a maximum dose of a composition to express and stimulate an opsin polypeptide, and/or to alter the length or timing of stimulation of the expressed opsin polypeptide in a subject (alone or in combination with other therapeutic agents) be used, that is, the highest safe dose or amount according to sound medical judgment. It will be understood by those of ordinary skill in the art, however, that a patient, also referred to herein as a subject, may insist upon a lower dose or tolerable dose for medical reasons, psychological reasons or for virtually any other reasons. Typically an effective amount of a treatment for a memory-impairment-associated disease or condition will be determined in clinical trials, establishing an effective dose for a test population versus a control population in a blind study.

A selected opsin polypeptide for use in methods of the invention may be administered using art-known methods. The manner and dosage administered may be adjusted by the individual physician, healthcare practitioner, or veterinarian, particularly in the event of any complication. The absolute amount administered will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Pharmaceutical compositions that include a fusion protein comprising a selected opsin polypeptide (or an encoding polynucleotide) for treatment methods of the invention may be administered to a subject: singly (alone), in combination with each other, and/or in combination with other drug therapies or other treatment regimens that are administered to the subject. A pharmaceutical composition used in the foregoing methods may contain an effective amount of a therapeutic compound (a selected stimulus-activated opsin polypeptide) that will increase the level of the opsin polypeptide to a level that when contacted with a suitable stimulus (e.g. illumination) parameters that will produce the desired response in a unit of weight or volume suitable for administration to a subject. In some embodiments of the invention, a pharmaceutical composition of the invention may include a pharmaceutically acceptable carrier.

Pharmaceutically acceptable carriers include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. In certain embodiments of the invention, such preparations may contain salt, buffering agents, preservatives, compatible carriers, aqueous solutions, water, etc. When used in medicine, the salts may be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

One or more of a selected stimulus-activated opsin polypeptide or encoding polynucleotide thereof, or a cell or vector comprising a nucleic acid sequence encoding a selected stimulus-activated opsin polypeptide, may be administered, for example in a pharmaceutical composition, directly to a cell or tissue in a subject. Direct tissue administration may be achieved by direct injection, and such administration may be done once, or alternatively a plurality of times. If administered two or more times, the polypeptides, polynucleotides, cells, and/or vectors may be administered via different routes. As a non-limiting example, a first (or the first few) administrations may be made directly into an affected tissue while later administrations may be into a different tissue.

A dose of a pharmaceutical composition of the invention that is administered to a subject to increase the level of a selected stimulus-activated opsin polypeptide in one or more cells of the subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The amount and timing of activation (also referred to herein as “stimulation”) of a selected opsin polypeptide delivered in a method of the invention (e.g., light wavelength, pulse length, length of light contact, duration of activation, intensity of light, etc.) that has been administered to a subject can also be adjusted based on efficacy of the treatment in a particular subject. Parameters for illumination and activation of a selected opsin delivered to a subject using a method of the invention, can be determined using teaching herein in conjunction with art-known methods, without requiring undue experimentation.

An embodiment of a treatment method of the invention may be administered to a subject under conditions suitable to increase dendritic spine density in one or a plurality of memory engram cells by inducing sufficient high-frequency neuronal firing in the memory engram cell to generate new dendritic spines. In certain aspects of the invention a treatment method is administered to a subject under conditions suitable to maintain a level of dendritic spine density in one or a plurality of memory engram cells by inducing sufficient high-frequency neuronal firing in the memory engram cell to maintain existing dendritic spines. In some aspects of the invention, a treatment method is administered to a subject under conditions suitable to both increase and maintain dendritic spine density in one or a plurality of memory engram cells by inducing sufficient high-frequency neuronal firing in the memory engram cell to generate new dendritic spines and maintain existing dendritic spines. Thus, different induction parameters can be used in a single subject at one or more different times.

It will be understood that a treatment method of the invention may be an acute treatment or a chronic treatment and that a subject may be administered one or both of an acute and chronic treatment, and that either type of treatment may be repeated one or more times in a subject. An acute treatment method of the invention may in some embodiments, comprise administering a selected light-activated opsin to predetermined cell type, (for example EC and/or DG cells), in a subject determined to be in need of treatment, contacting the opsin with light under suitable conditions to activate the opsin and induce high-frequency neuronal firing in one or a plurality of DG memory engram cells in the subject, thereby producing a temporally sustained increase in dendritic spines on the DG memory engram cells. An acute treatment method of the invention includes opsin activation conditions that result in a temporally sustained increase in dendritic spine density in one or a plurality of DG memory engram cells by means of one or both of: producing new dendritic spines on the DG memory engram cells and maintaining existing dendritic spines on the DG memory engram cells in the subject.

A chronic treatment method of the invention may in some embodiments, may comprise administrating a selected light-activated opsin to a predetermined cell type, (for example EC and/or DG cells) in a subject determined to be in need of treatment, contacting the opsin with light under suitable conditions 1, 2, 3, 4, 5, 6 7, 8, 9, 10, or more different times, to activate the opsin and induce high-frequency neuronal firing in one or a plurality of DG memory engram cells in the subject, thereby producing a temporally sustained increase in dendritic spines on the DG memory engram cells. A chronic treatment method of the invention includes opsin activation conditions that result in a temporally sustained increase in dendritic spine density in one or a plurality of DG memory engram cells by means of one or both of: producing new dendritic spines on the DG memory engram cells and maintaining existing dendritic spines on the DG memory engram cells in the subject.

In a chronic treatment of the invention, an interval of time between the end of one stimulation period of the light-activated opsin polypeptide and start of a subsequent stimulation period of the stimulus-activated opsin polypeptide is at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, one year, and two years, in length including all times within the stated range. In some aspects of the invention, the interval is over two years and in certain embodiments of the invention, the interval may be between 0.5 hours and 24 hours in length. It will be understood that the interval of time may be between a first and subsequent stimulation or may be between any two subsequent stimulations.

Aspects of methods of the invention include stimulating a selected stimulus-activated opsin polypeptide that is expressed in a cell, wherein the stimulation is under conditions sufficient to induce high-frequency neuronal firing in a DG cell in the subject, wherein the firing frequency is at least 45 Hz, 50 Hz, 60 Hz, 70 Hz, 80 Hz, 90 Hz, 100 Hz, 110 Hz, 120 Hz, 130 Hz, 140 Hz, 150 Hz, 160 Hz, 170 Hz, 180 Hz, 190 Hz, and 200 Hz. The frequency of the induced high-frequency neuronal firing may vary within a single stimulation period and may also vary between two more different stimulation periods. It will be understood that the frequency of firing may be less than 45 Hz at one or more time points within a period of stimulation, but a stimulation period in a method of the includes stimulation that induces neuronal firing at a frequency of at least 45 Hz, 50 Hz, 60 Hz, 70 Hz, 80 Hz, 90 Hz, 100 Hz, or 110 Hz and results in temporally sustained increase in dendritic spine density in one or a plurality of DG memory engram cells in a subject under treatment.

In acute and chronic treatment methods of the invention, the time duration of a stimulation period may vary in different embodiments of the invention. In some aspects of the invention, an optimal stimulation period may be determined for an individual subject by assessing memory before and after a treatment of the invention and/or may be based on clinical trials, and/or on stimulation period data from multiple subjects. An optimal stimulation period for a subject treated with a method of the invention may vary over time. For example, an initial stimulation period for a subject that expresses a selected light-activated opsin in a method of the invention may be one or more of: longer than, the same length as, and shorter than one or more subsequent stimulation periods in that subject. Thus, an initial stimulation period in a subject may be 1 hour, a second stimulation period in that subject may be 30 min., a third stimulation period in that subject may be 30 minutes, and a fourth stimulation period in that subject may be 2 hours. The length of a stimulation period may be determined for a given subject using art-known memory assessments, clinical trial data, or other means as described herein.

In some aspects of the invention the stimulation period is a time between 1 and 3 hours. In certain embodiments of the invention a stimulation period is at least one of between: 1 and 5 hours, 2 and five hours, 2 and 7 hours, 3 and 7 hours, 0.5 and 10 hours, 0.5 and 24 hours, 0.1 and 10 hours, 0.1 and 24 hours, 1 and 12 hours, 1 and 24 hours, 1 and 36 hours, 1 and 48 hours, and 1 and 60 hours, including all time points within the stated ranges. For example, in certain aspects of the invention, a time of a stimulation period of the stimulus-activated opsin polypeptide is at least 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 35, 40, 50, 60, 70, 80, 90, and 100 hours, including all numbers within the stated range.

It will be understood that a stimulation period and a frequency of high-frequency neuronal firing of a memory engram cells such as a DG cell, can be independently selected and optimized to result in a temporally sustained increase in dendritic spine density in one or more DG memory engram cells in a subject undergoing a treatment of the invention. As examples, though not intended to be limiting, the frequency of induced high-frequency neuronal firing is at least 90 Hz and the duration of the stimulation is between 1 and 3 hours; the frequency of induced high-frequency neuronal firing is at least 50 Hz and the duration of the stimulation is between 3 and 7 hours; the stimulation period and frequency of the induced high-frequency neuronal firing that results in a temporally sustained increase in dendritic spine density of a memory engram DG cell in a subject are at least 90 Hz and at least one hour, respectively; and the stimulation period and frequency of the induced high-frequency neuronal firing that results in a maintaining a temporally sustained increase in dendritic spine density of a memory engram DG cell in a subject are at least 50 Hz and at least one hour, respectively. Those in the art will be able to adjust the stimulation period and frequency of the induced high-frequency neuronal firing with routine procedures to assess and adjust as desirable for a subject's treatment.

In some aspects of the invention, a treatment is an “acute” treatment and in certain aspects of the invention a treatment is a “chronic” treatment. An acute treatment is a treatment of the invention in which a selected stimulus-activated opsin polypeptide is expressed in a predetermined cell type in a subject and the subject receives one stimulation period of the expressed opsin under conditions to induce a temporally-sustained increase in dendritic spine density in a memory engram cell. A chronic treatment of the invention may comprise two or more treatments administered to a subject over a period of time, wherein the two or more treatments may comprise one or more administrations of a selected stimulus-activated opsin polypeptide in a predetermined cell type in a subject, and two or more stimulation periods of expressed opsin under conditions suitable to induce a temporally-sustained increased dendritic spine density in a memory engram cell. Time intervals between two or more stimulation periods in a chronic treatment method of the invention are described elsewhere herein and may be adjusted as suitable for a subject undergoing chronic treatment.

In some circumstances one or more treatment methods may be combined with one or more additional treatments or changes in additional treatments administered to the subject to treat or assist in treating the memory-impairment-associated disease or condition. For example a treatment that is provided to a subject may be administered one or more of: prior to, concurrent with, or after additional treatments such as therapeutic agent, a behavior therapy, a cognitive therapy, a surgery, deep-brain stimulation, etc. A change or alteration in an additional treatment may include at least one of: starting, maintaining, increasing, reducing, or stopping administration of an additional therapeutic agent to the subject; starting, maintaining, increasing, reducing, or stopping administration of a behavioral therapy to the subject; starting, maintaining, increasing, reducing, or stopping administration of a deep brain stimulation therapy to the subject; administering a surgical therapy to the subject; starting, maintaining, increasing, reducing, and stopping administering a cognitive therapy to the subject; and starting, maintaining, increasing, reducing and stopping administering a counseling therapy to the subject. Non-limiting examples of therapeutic agents that a subject receiving a treatment method of the invention may also be administered, or have its administration be changed, include: a cholinesterase inhibitor, memantine, an antidepressant, an anxiolytic, and an antipsychotic. In embodiments of treatment methods of the invention, a means of selectively inducing high-frequency neuronal firing in one or more dentate gyrus (DG) memory engram cells does not include: deep brain stimulation and/or pharmacological induction.

### Administration Means

Various modes of administration known to the skilled artisan can be used to effectively deliver a pharmaceutical composition to increase the level of a stimulus-activated opsin polypeptide in a desired cell type and body region of a subject. Methods for administering such a pharmaceutical composition of the invention may be intravenous, intracavity, intrathecal, intrasynovial, intravitreal, trans-tissue, or other suitable means of administration. The invention is not limited by the particular modes of administration disclosed herein. Standard references in the art (e.g., Remington, The Science and Practice of Pharmacy, 2012, Editor: Allen, Loyd V., Jr, 22^{nd} Edition) provide modes of administration and formulations for delivery of various pharmaceutical preparations and formulations in pharmaceutical carriers. Numerous means for administrating of opsins to subjects and suitable parameters and methods for stimulating such opsins, are known and available in the art. Non-limiting examples of suitable methods with which to deliver fusion proteins into cells are presented herein and various methods are described in the art, [see for example: Klapoetke et al. (2014) Nature Methods 11(3), 338-346; for review see: Yizhar, O. et al. (2011) Neuron Vol. 71:9-34]. Art-known methods may be used to select and implement suitable stimulation parameters such as type of stimulation, illumination wavelength, intensity, illumination pulse rate, etc. for use with light-activated opsin expressed in cells and membranes in embodiments of methods of the invention. See for example: U.S. Pat. No. 8,957,028; U.S. Pat. No. 9,309,296; U.S. Pat. No. 9,284,353; U.S. Pat. No. 9,249,234; U.S. Pat. No. 9,101,690; PCT Pub. No. WO2013/07123; U.S. Pat Pub No. 20120214188; U.S. Pat Pub. No. 20160039902; U.S. Pat Pub No. 20140223679; Packer, A. M. et al., 2012 Nature Methods December 9(12):1202-1205; and Oron, D. et al., Progress in Brain Research, Chapter 7, Volume 196, 2012, Pages 119-143; the content of each of which is incorporated by references in its entirety herein. Methods and means for adjusting illumination variables and conditions such that they are suitable for activation of a light-activated opsin polypeptide are well-known in the art and representative methods can be found in publications such as: Lin, J., et al., Biophys. J. 2009 Mar. 4; 96(5):1803-14; Wang, H., et al., 2007 Proc Natl Acad Sci USA. 2007 May 8; 104(19):8143-8. Epub 2007 May 1; the contents of each of which is incorporated by reference in its entirety herein.

Other protocols that are useful for the administration of a pharmaceutical compound of the invention will be known to a skilled artisan, in which the dose amount, schedule of administration, sites of administration, mode of administration (e.g., intra-organ) and the like vary from those presented herein. Methods of delivering light-activated opsin molecules in vectors, and methods of expressing fusion proteins that include light-activated opsin molecules that are suitable for use in methods of the invention, include those described herein and other methods known in the art.

Administration of a cell or vector to increase expression of a stimulus-activated opsin polypeptide in one or more cells in a mammal other than a human; and methods to treat disorders or conditions, e.g. for testing purposes or veterinary therapeutic purposes, may be carried out under substantially the same conditions as described above. It will be understood that embodiments of the invention are applicable to both human and animals. Thus this invention is intended to be used in husbandry and veterinary medicine as well as in human therapeutics.

### Disorders, Diseases and Conditions

Methods of the invention may be used to express selected stimulus-activated opsin polypeptides to cells in subjects, and to activate the expressed opsins in a manner that alters voltage-associated cell activities. Such methods may be used to treat memory-impairment-associated disease and conditions such as Alzheimer's disease; dementia; brain injury; brain tumor; developmental delay; stroke; neurological disorder; Huntington's disease, learning disability; neurodegenerative disease; memory impairment due to substance abuse, poisoning, amnesia, trauma, Parkinson's disease, Creutzfeldt-Jakob disease, mild cognitive impairment, and depression. A memory-impairment associated disease may be at an early, mid, or late stage in a subject treated by a method of the invention. Assessment in a subject of the stage of a memory-impairment associated disease as early, mid, or late in the subject can be done using routine diagnostic and assessment methods.

It will be understood that the stage of a memory-impairment associated disorder or condition in a subject can be different at different times. For example, over time the status of a memory-impairment associated disease or condition in a subject may progress to a more severe later stage or may improve to a less advanced, less severe stage in the subject. In some aspects of the invention, treatment of a subject with a method of the invention results in a change in the stage of a memory-impairment associated disease or condition in the subject. For example, treatment of memory-impairment associated disease or condition in a subject with a method of the invention may reduce the severity of one or more symptoms and/or characteristics indicative of the disease or condition in the subject. In a non-limiting example, one or more symptoms of a memory-impairment associated disease or condition that are identified as present in a subject prior to treatment with a method of the invention, may change from a level indicative of a mid-stage of the disease or condition, to a level indicative of the early-stage of the disease or condition. In certain embodiments of the invention, one or more symptoms of a memory-impairment associated disease or condition that are present in a subject identified as being at an early stage of a memory-impairment associated disease or condition may be reduced due to treatment of the subject with a method of the invention.

In some aspects of the invention, compositions and methods of the invention, the term “treat” encompasses “augmenting” a condition that may not be considered to be a pathological condition. For example, a treatment method of the invention may be used in a subject who does not have a memory-impairment-associated disease or condition but rather with a desired goal of treating the subject to enhance memory and memory retrieval in the subject, wherein the subject is not considered to have a memory-impairment-associated disease or condition. For example, methods of the invention may be used to increase memory and memory retrieval from a level considered to near, at, or above a level considered “normal” and non-pathological. Thus, certain aspects of methods of the invention can be used to enhance and improve memory and memory retrieval ability in subjects not diagnosed with or suspected of having a memory-impairment-associated disease or condition, and that have a memory ability considered statistically near or at a normal level, or above a normal level.

In a non-limiting example, a treatment method of the invention comprises administering to a subject having, at risk of having, or suspected of having Alzheimer's disease, a fusion protein comprising a selected light-activated opsin polypeptide and the expressed selected opsin polypeptide is activated with illumination under suitable conditions to induce high-frequency neuronal firing in a memory engram cell, such as a DG cell in the subject. In another non-limiting example, a treatment method of the invention comprises administering to a subject having, at risk of having, or suspected of having a traumatic brain injury, a fusion protein comprising a selected light-activated opsin polypeptide and the expressed selected opsin polypeptide is activated with illumination under suitable conditions to induce high-frequency neuronal firing in a memory engram cell, such as a DG cell in the subject. In some embodiments of the invention, a fusion protein comprising a selected light-activated opsin polypeptide is administered to a subject who has, or is suspected of having dementia and the selected opsin polypeptide is activated in a suitable manner to induce high-frequency neuronal firing of memory engram DG cells in the subject as a treatment for the dementia. In some embodiments of the invention, a fusion protein comprising a selected light-activated opsin polypeptide is administered to a subject and activated in a manner that induces high-frequency neuronal firing in memory engram DG cells in the subject, wherein the subject is also treated for the memory-impairment-associated disease or condition at least one of before, during, and after the treatment of the invention with one or more of: a therapeutic agent and behavioral treatment designed to treat the memory-impairment-associated disease or condition. Overall, methods and therapeutic compositions have now been identified that are useful to increase and maintain dendritic spine density in memory engram cells such as DG cells, and such methods can be used to improve memory and memory retrieval ability in subjects and (1) treat memory-impairment-associated disease and conditions and (2) improve normal or near-normal memory.

### Testing Methods

As a non-limiting example, a treatment method of the invention can be used at a time that is one or more of: before, during, or after administration of a candidate therapeutic agent or compound that is tested to see if it augments the treatment of the invention, inhibits efficacy of the treatment of the invention, or is synergistic with a treatment method of the invention. In such methods of the invention, additional and combination treatments for diseases, disorders, or conditions can be assessed and efficacy determined. In one embodiment of the invention, in a subject, a test cell in which a fusion protein comprising a selected light-activated opsin polypeptide is expressed according to a method of the invention is contacted with a light that depolarizes the cell or otherwise alters ion flux across the cell membrane and the subject is also administered a candidate compound. The cell and/or subject that include the cell can be monitored for the presence or absence of a change that occurs in test conditions versus a control condition. For example, in a cell, an activity increase in the test cell may be a change in the depolarization of the test cell, a change in one or more of the subject's memory, memory retrieval, and another characteristic of a disease or condition being treated with the method of the invention. Art-known methods can be used to assess electrical activity and ion flux activity and changes in memory and memory retrieval with or without additional contact with a candidate compound and such results can be compared to assess the effect of the candidate compound.

### Kits

The invention, in part also includes kits that can be used in treatment methods of the invention. Such kits may comprise one or more of: a polynucleotide that encodes a selected stimulus-activated opsin polypeptide, vectors, additional components to include in vectors, cells, control vectors, control compounds, etc. A kit may also include instructions for delivering a treatment method of the invention to a subject comprising one or more of: expressing a selected stimulus-activated opsin polypeptide in a predetermined cell in a subject; contacting the expressed opsin with a light suitable to induce high-frequency neuronal firing in a plurality of memory engram DG cells resulting in a temporally sustained increase in dendritic spine density on one or more of the plurality of memory engram DG cells.

### EXAMPLES

### Example 1

Studies were performed to assess memory retrieval resulting from activation of memory engram cells and use of such methods to treat Alzheimer's disease and memory-impairment-associated diseases and conditions. Addition information is provided in Roy, D. S. et al. (2016) Nature Vol. 531:508-512 and Extended Data section; the content of which is incorporated herein by reference in its entirety.

### Subjects.

The APP/PS1 double-transgenic AD mice [Jankowski, J. L., et al. (2004) Hum. Mol. Genet. 13, 159-170], originally described as Line 85, were obtained from Jackson Laboratory, Bar Harbor, Me. (stock number 004462). Under the control of mouse prion promoter elements, these mice express a chimeric mouse/human APP transgene containing Swedish mutations (K595N/M596L) as well as a mutant human PS1 transgene (delta exon 9 variant). To label memory engram cells in APP/PS1 mice, a triple transgenic mouse line was generated by mating c-Fos.tTA [Liu, X., et al. (2012) Nature 484, 381-385], [Reijmers, L. G., et al. (2007) Science 317, 1230-1233] transgenic mice with APP/PS1 double-transgenic mice. The PS1/APP/tau [Oddo, S., et al. (2003) Neuron 39, 409-421] triple-transgenic AD mice were obtained from Jackson Laboratory (stock number 004807). These 3×Tg-AD mice express a mutant human PS1 transgene (M146V), a human APP transgene containing Swedish mutations (KM670/671NL) and a human MAPTtransgene harboring the P301L mutation. All mouse lines were maintained as hemizygotes. Mice had access to food and water ad libitum and were socially housed in numbers of two to five littermates until surgery. After surgery, mice were singly housed. For behavioral experiments, all mice were male and 7-9 months old. For optogenetic experiments, mice had been raised on food containing 40 mgkg⁻¹ DOX for at least 1 week before surgery, and remained on DOX for the remainder of the experiments except for the target engram labelling days. For in vitro electrophysiology experiments, mice were 24-28 days old at the time of surgery. All experiments were conducted in accordance with US National Institutes of Health (NIH) guidelines and the Massachusetts Institute of Technology Department of Comparative Medicine and Committee of Animal Care. No statistical methods were used to predetermine sample size.

### Viral Constructs.

A previously established method [Liu, X., et al. (2012) Nature 484, 381-385] for labelling memory engram cells combined c-Fos-tTA transgenic mice with a DOX-sensitive adeno-associated virus (AAV). However, in this study, the method was modified using a double virus system to label memory engram cells in the early AD mice, which already carry two transgenes. The pAAV-c-Fos-tTA plasmid was constructed by cloning a 1 kb fragment from the c-Fos gene (550 bp upstream of c-Fos exon I to 35 bp into exon II) into an AAV backbone using the KpnI restriction site at the 5′ terminus and the SpeI restriction site at the 3′ terminus. The AAV backbone contained the tTA-Advanced [Urlinger, S., et al. (2000) Proc. Natl. Acad. Sci USA 97, 7063-7968] sequence at the SpeI restriction site. The pAAV-TRE-ChR2-eYFP and pAAV-TRE-eYFP constructs have been described previously [Liu, X., et al. (2012) Nature 484, 381-385][Ramirez, S., et al. (2013) Science 341, 387-391]. The pAAV-TRE-oChIEF-tdTomato [Lin, J. Y., et al. (2009) Biophys. J. 96, 1803-1814] plasmid was constructed by replacing the ChR2-eYFP fragment from pAAV-TRE-ChR2-eYFP plasmid using NheI and MfeI restriction sites. The pAAV-CaMKII-oChIEF-tdTomato plasmid was constructed by replacing the TRE fragment from the pAAV-TRE-oChIEF-tdTomato plasmid using BamHI and EcoRI restriction sites. The pAAV-TRE-DTR-eYFP [Zhan, C. et al (2013) J. Neurosci. 33, 3624-3632] plasmid was constructed by replacing the ChR2 fragment from the pAAV-TRE-ChR2-eYFP plasmid using EcoRI and AgeI restriction sites. AAV vectors were serotyped with AAV₉ coat proteins and packaged at the University of Massachusetts Medical School Gene Therapy Center and Vector Core. Viral titres were 1.5×10¹³ genome copy (GC) ml⁻¹ for AAV₉-c-Fos-tTA, AAV₉-TRE-ChR2-eYFP and AAV₉-TRE-eYFP, 1×10¹³ GC ml⁻¹ for AAV₉.TRE-oChIEF-tdTomato, 4×10¹³ GCml⁻¹ for AAV₉-CaMKII-oChIEF-tdTomato and 2×10¹³ GC ml⁻¹ for AAV₉-TRE-DTR-eYFP.

### Surgery and Optic Fiber Implants.

Mice were anaesthetized with isoflurane or 500 mg kg⁻¹ avertin for stereotaxic injections [Ryan, T. J., et al. (2015) Science 348, 1007-1013]. Injections were targeted bilaterally to the DG (−2.0 mm anteroposterior (AP), ±1.3 mm mediolateral (ML), −1.9 mm dorsoventral (DV)), MEC (−4.7 mm AP, ±3.35 mm ML, −3.3 mm DV) and LEC (−3.4 mm AP, ±4.3 mm ML, −4.0 mm DV). Injection volumes were 300 nl for DG and 400 nl for MEC and LEC. Viruses were injected at 70 nl min⁻¹ using a glass micropipette attached to a 10 ml Hamilton microsyringe. The needle was lowered to the target site and remained for 5 min before beginning the injection. After the injection, the needle stayed for 10 min before it was withdrawn. A custom DG implant containing two optic fibers (200 mm core diameter; Doric Lenses) was lowered above the injection site (−2.0 mm AP, ±1.3 mm ML, −1.7 mm DV). The implant was secured to the skull with two jewelry screws, adhesive cement (C&B Metabond), and dental cement. An opaque cap derived from the top part of an Eppendorf tube protected the implant. Mice were given 1.5 mg kg⁻¹ metacam as analgesic and allowed to recover for 2 weeks before behavioral experiments. All injection sites were verified histologically. As criteria, only mice with virus expression limited to the targeted region were included.

### Systemic Injection of Kainic Acid.

For seizure experiments [Liu, X., et al. (2012) Nature 484, 381-385], mice were taken off DOX for 1 day and injected intraperitoneally with 15 mg kg⁻¹ kainic acid (KA). Mice were returned to DOX food 6 h after KA treatment and perfused the next day for immunohistochemistry procedures.

### Immunohistochemistry.

Mice were dispatched using 750-1,000 mg kg⁻¹ avertin and perfused transcardially with PBS, followed by 4% paraformaldehyde (PFA). Brains were extracted and incubated in 4% PFA at room temperature overnight. Brains were transferred to PBS and 50 μm coronal slices were prepared using a vibratome. For immunostaining [Ryan, T. J., et al. (2015) Science 348, 1007-1013], each slice was placed in PBS+0.2% Triton X-100 (PBS-T), with 5% normal goat serum for 1 h and then incubated with primary antibody at 4° C. for 24 h. Slices then underwent three wash steps for 10 min each in PBS-T, followed by 1 h incubation with secondary antibody. After three more wash steps of 10 min each in PBS-T, slices were mounted on microscope slides. All analyses were performed blind to the experimental conditions. Antibodies used for staining were as follows: to stain for ChR2-eYFP, DTR-eYFP or eYFP alone, slices were incubated with primary chicken anti-GFP (1:1,000, Life Technologies) and visualized using anti-chicken Alexa-488 (1:200). For plaques, slices were stained using primary mouse anti-β-amyloid (1:1,000; Sigma-Aldrich) and secondary anti-mouse Alexa-488 (1:500). c-Fos was stained with rabbit anti-c-Fos (1:500, Calbiochem) and anti-rabbit Alexa-568 (1:300). Adult newborn neurons were stained with guinea pig anti-DCX (1:1,000; Millipore) and anti-guinea-pig Alexa-555 (1:500). Neuronal nuclei were stained with mouse anti-NeuN (1:200; Millipore) and Alexa-488 (1:200). DG mossy cell axons were stained with mouse anti-CR (1:1,000; Swant) and Alexa-555 (1:300).

### Cell Counting.

To characterize the expression pattern of ChR2-eYFP, DTR-eYFP, eYFP alone and oChIEF-tdTomato in control and AD mice, the number of eYFP⁺/tdTomato⁺ neurons were counted from 4-5 coronal slices per mouse (n=3-5 mice per group). Coronal slices centered on coordinates covered by optic fiber implants were taken for DG quantification and sagittal slices centered on injection coordinates were taken for MEC and LEC. Fluorescence images were acquired using a Zeiss Axiolmager.Z1/ApoTome microscope (×20). Automated cell counting analysis was performed using ImageJ software. The cell body layers of DG granule cells (upper blade), MEC or LEC cells were outlined as a region of interest (ROI) according to the DAPI signal in each slice. The number of eYFP⁺/tdTomato⁺ cells per section was calculated by applying a threshold above background fluorescence. Data were analyzed using Microsoft Excel with the Statplus plug-in. A similar approach was applied for quantifying amyloid-3 plaques, c-Fos⁺ neurons and adult newborn (DCX⁺) neurons. Total engram cell reactivation was calculated as ((c-Fos⁺ eYFP⁺)/(total DAPI⁺))×100. Chance overlap was calculated as ((c-Fos⁺/total DAPI⁺)×(eYFP⁺/total DAPI⁺))×100. Percentage of adult newborn neurons expressing neuronal markers was calculated as ((NeuN⁺ DCX⁺)/(total DCX⁺))×100. DAPI⁺ counts were approximated from five coronal/sagittal slices using ImageJ. All counting experiments were conducted blind to experimental group. A first researcher (Researcher 1) trained the animals, prepared slices and randomized images, while a second research (researcher 2) performed semi-automated cell counting. Statistical comparisons were performed using unpaired t-tests: *P<0.05, **P<0.01, ***P<0.001.

### Spine Density Analysis.

Engram cells were labelled using c-Fos-tTA-driven synthesis of ChR2-eYFP or eYFP alone. The eYFP signal was amplified using immunohistochemistry procedures, after which fluorescence z-stacks were taken by confocal microscopy (Zeiss LSM700) using a×40 objective. Maximum intensity projections were generated using ZEN Black software (Zeiss). Four mice per experimental group were analyzed for dendritic spines. For each mouse, 30-40 dendritic fragments of 10-μm length were quantified (n=120-160 fragments per group). To measure spine density of DG engram cells with a focus on entorhinal cortical inputs, distal dendritic fragments in the middle-to-outer molecular layer (ML) were selected. For CA3 and CA1 engram cells, apical and basal dendritic fragments were selected. To compute spine density, the number of spines counted on each fragment was normalized by the cylindrical approximation of the surface of the specific fragment. Experiments were conducted blind to experimental group. Researcher 1 imaged dendritic fragments and randomized images, while researcher 2 performed manual spine counting.

### In Vitro Recordings.

After isoflurane anesthesia, brains were quickly removed and used to prepare sagittal slices (300 μm) in an oxygenated cutting solution at 4° C. with a vibratome [Ryan, T. J., et al. (2015) Science 348, 1007-1013]. Slices were intubated at room temperature in oxygenated artificial cerebrospinal fluid (ACSF) until the recordings. The cutting solution contained (in mM): 3 KCl, 0.5 CaCl₂, 10 MgCl₂, 25 NaHCO₃, 1.2 NaH₂PO₄, 10_{D}-glucose, 230 sucrose saturated with 95% O₂-5% CO₂ (pH 7.3, osmolarity of 340 mOsm). The ACSF contained (in mM): 124 NaCl, 3 KCl, 2 CaCl₂, 1.3MgSO₄, 25 NaHCO₃, 1.2 NaH₂PO₄, 10_{D}-glucose, saturated with 95% O₂-5% CO₂ (pH 7.3, 300 mOsm). Individual slices were transferred to a submerged experimental chamber and perfused with oxygenated ACSF warmed at 35° C. (±0.5° C.) at a rate of 3 ml min¹ during recordings. Current or voltage clamp recordings were performed under an IR-DIC microscope (Olympus) with a×40 water immersion objective (0.8 NA), equipped with four automatic manipulators (Luigs & Neumann) and a CCD camera (Hamamatsu). Borosilicate glass pipettes (Sutter Instruments) were fabricated with resistances of 8-10 MΩ. The intracellular solution (in mM) for current clamp recordings was: 110 K-gluconate, 10 KCl, 10 HEPES, 4 ATP, 0.3 GTP, 10 phosphocreatine, 0.5% biocytin (pH 7.25, 290 mOsm). Recordings used two dual channel amplifiers (Molecular Devices), a 2 kHz filter, 20 kHz digitization and an ADC/DAC data acquisition unit (Instrutech) running on custom software in Igor Pro (Wavemetrics). Data acquisition was suspended whenever the resting membrane potential was depolarized above −50 mV or the access resistance (RA) exceeded 20 MO. Optogenetic stimulation was achieved using a 460 nm LED light source (Lumen Dynamics) driven by TTL input with a delay onset of 25 μs (subtracted offline for latency estimation). Light power on the sample was 33 mW mm⁻². To test oChIEF expression, EC cells were stimulated with a single light pulse of 1 s, repeated 10 times every 5 s. DG granule cells were held at −70 mV. Optical LTP protocol: 5 min baseline (10 blue light pulses of 2 ms each, repeated every 30 s) was acquired before the onset of the LTP protocol (100 blue light pulses of 2 ms each at a frequency of 100 Hz, repeated 5 times every 3 min) and the effect on synaptic amplitude was recorded for 30 min (1 pulse of 2 ms every 30 s). Using the 5 min baseline recording data, EPSPs were normalized (FIG. 3J). Potentiation was observed in 6 out of 30 cells and results were statistically confirmed using a two tailed paired t-test. Experiments were performed in the presence of 10 M gabazine (Tocris) and 2 M CGP55845 (Tocris). Recorded cells were recovered for morphological identification using streptavidin CF633 (Biotium).

### In Vivo Recordings.

Multi-unit responses to optical stimulation were recorded in the DG of mice injected with a cocktail of AAV₉-c-Fos-tTA and AAV₉.TRE-oChIEF-tdTomato viruses into MEC/LEC. Mice were anaesthetized (10 ml kg⁻¹) using a mixture of ketamine (100 mg ml¹)/xylazine (20 mg ml⁻¹) and placed in the stereotactic system. Anesthesia was maintained by booster doses of ketamine (100 mg kg⁻¹). An optrode consisting of a tungsten electrode (0.5 MΩ) attached to an optic fiber (200-μm core diameter), with the tip of the electrode extending beyond the tip of the fiber by 300 μm, was used for simultaneous optical stimulation and extracellular recording. The power intensity of light emitted from the optrode was calibrated to about 10 mW, consistent with the power used in behavioral assays. oChIEF⁺ cells were identified by delivering 20-ms light pulses (1 Hz) to the recording site every 50-100 m. After light responsive cells were detected, multi-unit activity in response to trains of light pulses (200 ms) at 100 Hz was recorded. Data acquisition used an Axon CNS Digidata 1440A system. MATLAB analysis was performed, as previously described [Ramirez, S., et al. (2013) Science 341, 387-391].

### Behavior Assays.

Experiments were conducted during the light cycle (7 a.m. to 7 p.m.). Mice were randomly assigned to experimental groups for specific behavioral assays immediately after surgery. Mice were habituated to investigator handling for 1-2 min on three consecutive days. Handling took place in the holding room where the mice were housed. Before each handling session, mice were transported by wheeled cart to and from the vicinity of the behavior rooms to habituate them to the journey. For natural memory recall sessions, data were quantified using FreezeFrame software. Optogenetic stimulation interfered with the motion detection, and therefore all light-induced freezing behavior was manually quantified. All behavior experiments were analyzed blind to experimental group. Unpaired Student's t-tests were used for Independent group comparisons, with Welch's correction when group variances were significantly different. Given behavioral variability, initial assays were performed using a minimum of 10 mice per group to ensure adequate power for any observed differences. Experiments that resulted in significant behavioral effects were replicated three times in the laboratory. Following behavioral protocols, brain sections were prepared to confirm efficient viral labelling in target areas. Animals lacking adequate labelling were excluded before behavior quantification.

### Contextual Fear Conditioning.

Two distinct contexts were employed [Ryan, T. J., et al. (2015) Science 348, 1007-1013]. Context A was 29×25×22 cm chambers with grid floors, opaque triangular ceilings, red lighting, and scented with 1% acetic acid. Four mice were run simultaneously in four identical context A chambers. Context B consisted of four 30×25×33 cm chambers with perspex floors, transparent square ceilings, bright white lighting, and scented with 0.25% benzaldehyde. All mice were conditioned in context A (two 0.60 mA shocks of 2 s duration in 5 min), and tested (3 min) in contexts A and B 1 day later. Experiments showed no generalization in the neutral context B. All experimental groups were counter-balanced for chamber within contexts. Floors of chambers were cleaned with quatricide before and between runs. Mice were transported to and from the experimental room in their home cages using a wheeled cart. The cart and cages remained in an anteroom to the experimental rooms during all behavioral experiments. For engram labeling, mice were kept on regular food without DOX for 24 h before training. When training was complete, mice were switched back to food containing 40 mg kg⁻¹ DOX.

### Open Field.

Spontaneous motor activity was measured in an open field arena (56×26 cm) for 10 min. All mice were transferred to the testing room and acclimated for 30 min before the test session. During the testing period, lighting in the room was turned off. The apparatus was cleaned with quatricide before and between runs. Total movements (distance travelled and velocity) in the arena were quantified using an automated infrared (IR) detection system (EthoVision XT, Noldus). The tracking software plotted heat maps for each mouse, which was averaged to create representative heat maps for each genotype. Raw data were extracted and analyzed using Microsoft Excel.

### Engram Activation.

For light-induced freezing behavioral context distinct from the CFC training chamber (context A) was used. These were 30×25×33 cm chambers with perspex floors, square ceilings, white lighting, and scented with 0.25% benzaldehyde. Chamber ceilings were customized to hold a rotary joint (Doric Lenses) connected to two 0.32-m patch cords. All mice had patch cords fitted to the optic fiber implant before testing. Two mice were run simultaneously in two identical chambers. ChR2 was stimulated at 20 Hz (15 ms pulse width) using a 473 nm laser (10-15 mW), for the designated epochs. Testing sessions were 12 min in duration, consisting of four 3 min epochs, with the first and third as light-off epochs, and the second and fourth as light-on epochs. At the end of 12 min, the mouse was detached and returned to its home cage. Floors of chambers were cleaned with quatricide before and between runs.

### In Vivo Optical LTP.

One day after CFC training and engram labelling (DG plus PP terminals) in control and early AD groups, mice were placed in an open field arena (52×26 cm) after patch cords were fitted to the fiber implants. After a 15 min acclimatization period, mice with oChIEF⁺ PP engram terminals in the DG received the optical LTP [Nabavi, S., et al. (2014) Nature 511, 349-352] protocol (100 blue light pulses of 2 ms each at a frequency of 100 Hz, repeated 5 times every 3 min). This in vivo protocol was repeated 10 times over a 3 h duration. After induction, mice remained in the arena for an additional 15 min before returning to their home cage. To apply optical LTP to a large portion of excitatory MEC neurons, an AAV virus expressing oChIEF-tdTomato under the CaMKII promoter, rather than a c-Fos-tTA/TRE virus (that is, engram labelling), was used. For protein synthesis inhibition experiments, immediately after the in vivo LTP induction protocol mice received 75 mg kg⁻¹ anisomycin (Aniso) or an equivalent volume of saline intraperitoneally. Mice were then returned to their home cages. An hour later, a second injection of Aniso or saline was delivered.

### Inhibitory Avoidance.

A 30×28×34 cm unscented chamber with transparent square ceilings and intermediate lighting was used. The chamber consisted of two sections, one with grid flooring and the other with a white light platform. During the conditioning session (1 min), mice were placed on the light platform, which is less preferred section of the chamber (relative to the grid section). Once mice entered the grid section of the chamber (all four feet), 0.80 mA shocks of 2 s duration were delivered. On average, each mouse received 2-3 shocks per training session. After 1 min, mice were returned to their home cage. The next day, latency to enter the grid section of the chamber as well as total time on the light platform was measured (3 min test).

### Novel Object Location.

Spatial memory was measured in a white plastic chamber (28×28 cm) that had patterns (series of parallel lines or circles) on opposite walls. The apparatus was unscented and intermediate lighting was used. All mice were transferred to the behavioral room and acclimated for 30 min before the training session. On day 1, mice were allowed to explore the chamber with patterns for 15 min. On days 2 and 3, mice were introduced into the chamber that had an object (7-cm-tall glass flask filled with metal beads) placed adjacent to either patterned wall. The position of the object was counter-balanced within each genotype. On day 4, mice were placed into the chamber with the object either in the same position as the previous exposure (familiar) or at a novel location based on wall patterning. Frequency of visits to the familiar and novel object locations was quantified using an automated detection system (EthoVision XT, Noldus). Total time exploring the object was also measured (nose within 1.5 cm of object). The tracking software plotted heat maps based on exploration time, which was averaged to create representative heat maps for each genotype. Raw data were extracted and analyzed using Microsoft Excel.

### Results

A mouse model of Alzheimer's disease (herein also referred to as ‘AD mice’) over-expresses the delta exon 9 variant of presenilin 1 (PS1; also known as PSENI), in combination with the Swedish mutation of β-amyloid precursor (APP). 9-month-old AD mice showed severe plaque deposition across multiple brain regions (FIG. 1A), specifically in the dentate gyrus (DG) (FIG. 1B) and medial entorhinal cortex (EC) (FIG. 1E); in contrast, 7-month-old AD mice lacked amyloid plaques (FIG. 1D and FIG. 5A-D). Focusing on these two age groups of AD mice, short-term (1 h; STM) and long-term (24 h; LTM) memory formation was quantified using contextual fear conditioning (CFC) (FIG. 1E). Nine-month-old AD mice were impaired in both STM and LTM, which suggested a deficit in memory encoding (FIG. 1K-O). By contrast, 7-month old AD mice showed normal levels of training-induced freezing (FIG. 1F) and normal STM (FIG. 1G), but were impaired in LTM (FIG. 1H). Neither control nor 7-month-old AD mice displayed freezing behavior in a neutral context (FIG. 1I). In the DG of 7 month-old AD mice, the levels of cells that were immediate early gene c-Fos-positive after CFC training were normal, but were lower compared with control mice after LTM tests (FIG. 1J). Motor behaviors and the density of DG granule cells were normal in these mice (FIG. 5E-K). Thus, these behavioral- and cellular-level observations confirmed that 7-month-old AD mice serve as a mouse model of early AD regarding memory impairments.

Recently, molecular, genetic and optogenetic methods to identify neurons that hold traces, or engrams, of specific memories have been established [Liu, X., et al. (2012) Nature 484, 381-385] [Ramirez, S., et al. (2013) Science 341, 387-391]. Using this technology, several groups have demonstrated that DG neurons activated during CFC learning are both sufficient and necessary for subsequent memory retrieval [Liu, X., et al. (2012) Nature 484, 381-385][Ramirez, S., et al. (2013) Science 341, 387-391] [Redondo, R. L., et al. (2014) Nature 513, 426-430] [Ryan, T. J., et al. (2015) Science 348, 1007-1013] [Denny, C. A., et al. (2014) Neuron 83, 189-201]. In addition, a recent study found that engram cells under protein-synthesis-inhibitor-induced amnesia were capable of driving acute memory recall if they were directly activated optogenetically [Ryan, T. J., et al. (2015) Science 348, 1007-1013]. Here, the memory engram cell identification and manipulation technology was applied to 7-month-old AD mice to determine whether memories could be retrieved in the early stages of the disease. Because the EC-hippocampus (HPC) network is among the earliest to show altered synaptic/dendritic properties and these alterations have been suggested to underlie the memory deficits in early AD [Harris, J. A., et al. (2010) Neuron 68, 428-441] [Hyman, B. T., et al. (1986) Ann Neurol. 20, 472-481], there was a focus on labelling the DG component of CFC memory engram cells of 7 month-old AD mice using a double adeno-associated virus (AAV) system (FIG. 1P-Q). Although on a doxycycline (DOX) diet DG neurons completely lacked channelrhodopsin 2 (ChR2)-enhanced yellow fluorescent protein (eYFP) labelling, 1 day off DOX was sufficient to permit robust ChR2-eYFP expression in control mice (FIGS. 1R-S and FIGS. 6A-C), as well as in 7-month old AD mice (FIG. 1T-U).

As expected, these engram-labelled early AD mice were amnesic a day after CFC training (FIG. 1V). But, remarkably, these mice froze on the next day in a distinct context (context B) as robustly as equivalently treated control mice in response to blue light stimulation of the engram cells (FIG. 1W). This light-specific freezing was not observed using on DOX mice (FIG. 6D-O). A natural recall test conducted on the third day in the conditioning context (context A) revealed that the observed optogenetic engram reactivation did not restore memory recall by natural cues in early AD mice (FIG. 1X). This was the case even after multiple rounds of light activation of the engram cells (FIG. 7A-C). Experiments were performed to replicate the successful optogenetic rescue of memory recall in two other models of early AD: a triple transgenic line obtained by mating c-Fos-tTA mice with double-transgenic APP/PS1 mice (FIG. 8A-G) and a widely used triple-transgenic AD model [Oddo, S., et al. (2003) Neuron 39, 409-421] (PS1/APP/tau (also known as MAPT); FIG. 8H-M). These data showed that DG engram cells in 7-month-old mouse models of early AD are sufficient to induce memory recall upon optogenetic reactivation, which indicates a deficit of memory retrievability during early AD-related memory loss.

An age-dependent decrease was detected (FIG. 9A) in dendritic spine density of DG engram cells in early AD mice (FIG. 2A-C), showing that the long-term memory impairments of early AD correlate with dendritic spine deficits of DG engram cells (FIG. 9B). The inability to generate newborn neurons in the DG could play apart in the development of AD-specific cognitive deficits [Rodriguez, J. J., et al. (2008) PLoS One 3, e2935]. However, early AD mice showed similar levels of neurogenesis in the DG compared with control mice, which were quantified using doublecortin (DCX) staining (FIG. 5L-Q). Putative CFC memory engram cells were labeled in both medial EC (MEC) and lateral EC (LEC) with oChlEF [Lin, J. Y., et al. (2009) Biophys. J. 96, 1803-1814] (a variant of ChR2) and, simultaneously, CFC memory engram cells were labelled in the DG with eYFP (FIG. 2D). With this procedure, perforant path (PP) terminals are also labelled with oChIEF (FIG. 2E-F). One day after footshocks, these terminals were optogenetically activated and quantified the overlap between putative DG engram cells (that is, eYFP⁺, green) and DG cells in which the endogenous c-Fos (red) had been activated by the optogenetic activation of oChIEF⁺ PP terminals. Both control and early AD mice showed above-chance and indistinguishable levels of c-Fos⁺/eYFP⁺ overlap, indicating that the preferential functional connectivity between engram cells is maintained in the early AD mice (FIG. 2G-I).

Experiments were performed to assess whether the reversal of dendritic spine deficits in DG engram cells of early AD mice would rescue long-term memory. To investigate this possibility, studies were performed to assess whether long-term potentiation (LTP) induced in vivo with light, using oChIEF [Nabavi, S., et al. (2014) Nature 511, 348-352] would result in increased dendritic spine density. Learning-dependent labelling was validated, with oChIEF, of neurons in the MEC (FIG. 3A-C and FIG. 10A-C) and LEC (FIG. 3D) as well as PP terminals in the DG (FIG. 3E-F). In vivo extracellular recording upon light stimulation of oChIEF⁺ EC axonal terminals in the DG showed a reliable spiking response of DG cells in anaesthetized control mice (FIG. 3G). Furthermore, a previously established optical LTP protocol was utilized in HPC slices from control mice to induce LTP in DG cells (FIG. 3H-J) [Nabavi, S., et al. (2014) Nature 511, 348-352]. These biocytin-filled DG cells revealed an increase in spine density after in vitro optical LTP (FIG. 10D).

In early AD mice, in vivo application of the engram-specific optical LTP protocol restored spine density of DG engram cells to control levels (AD+100 Hz group; FIG. 3K-L). Furthermore, this spine restoration in early AD mice correlated with amelioration of long-term memory impairments observed during recall by natural cues (FIG. 3M), an effect that persisted for at least 6 days after training (AD rescue+diphtheria toxin receptor (DTR)+saline group; FIG. 3P). The LTP-induced spine restoration and behavioral deficit rescue were protein-synthesis dependent (FIG. 11A-B). The rescued memory was context-specific (FIG. 12A). In addition, long-term memory recall of age-matched control mice was unaffected by this optical LTP protocol (FIG. 12B). By contrast, applying the optical LTP protocol to a large portion of excitatory PP terminals in the DG (that is, with no restriction to the PP terminals derived from EC engram cells) did not result in long-term memory rescue in early AD mice (FIG. 13A-F). To confirm the correlation between restoration of spine density of DG engram cells and amelioration of long-term memory impairments, which were both induced by the optical LTP protocol, comparisons were made between the overlap of natural-recall-cue-induced c-Fos⁺ cells and CFC-training-labeled DG engram cells after an application of the engram-specific LTP protocol to early AD mice (FIG. 3N). Early AD mice that did not receive the optical LTP protocol showed low levels of c-Fos⁺/eYFP⁺ overlap compared with control mice upon natural recall cue delivery. By contrast, early AD mice that went through the optical LTP protocol showed c-Fos⁺/eYFP⁺ overlap similar to that of control mice (FIG. 3N). Thus, these data suggest that spine density restoration in DG engram cells contributes to the rescue of long term memory in early AD mice.

Because of the highly redundant connectivity between the EC and DG [Tamamaki, N. & Nojyo, Y. (1993) Hippocampus 3, 471-480], it is possible that the extensive optical LTP protocol also augmented spine density in some non-engram DG cells. To establish a link between the spine rescue in DG engram cells and the behavioral rescue of early AD mice, an engram-specific ablation [Zhan, C., et al. (2013) J. Neurosci. 33, 3624-3632] virus was developed. It was confirmed that this DTR-mediated method efficiently ablated DG engram cells after diphtheria toxin (DT) administration (FIG. 3O), while leaving the nearby DG mossy cells intact (FIG. 14A-E). By simultaneously labelling axonal terminals of PP with oChIEF and DG engram cells with DTR, the effect of DG engram cell ablation after optical LTP-induced behavioral rescue was examined (FIG. 3P). Within-animal comparisons (test 1 versus test 2) showed a decrease in freezing behavior of LTP-rescued AD mice in which DG engram cells were ablated. These data strengthen the link between DG engram cells with restored spine density and long-term behavioral rescue in early AD mice.

To examine whether the optical LTP-induced behavioral rescue could be applied to DG engram cells from other learning experiences, memory engrams for inhibitory avoidance or novel object location in early AD mice were labelled (FIG. 4A). Early AD mice showed memory impairments in inhibitory avoidance memory and novel object location spatial memory (FIG. 4B-C). Optical LTP-induced spine rescue at the PP-DG engram synapses was sufficient to reverse long-term memory impairments of early AD mice in both behavioral paradigms, thus demonstrating the versatility of this engram-based intervention.

The studies described herein demonstrated that optogenetic activation of HPC cells active during learning elicits memory recall in mouse models of early AD. These results are believed to be the first rigorous demonstration that memory failure in early AD models reflects an impairment in the retrieval of information. Further support for a memory retrieval impairment in early AD comes from the fact that impairments are in LTM (at least 1 day long), but not in STM (˜1 h after training), which is consistent with a retrieval deficit. The retrieval deficit in early AD models is similar to memory deficits observed in amnesia induced by impairing memory consolidation via protein synthesis inhibitors [Ryan, T. J., et al. (2015) Science 348, 1007-1013].

The conclusions based on the studies described herein apply to episodic memory, which involves processing by HPC and other medial temporal lobe structures. The results contribute to a better understanding of memory retrieval deficits in several cases of early AD, and may apply to other pathological conditions, such as Huntington's disease [Hodges, J. R. et al. (1990) J. Neurol. Neurosurg. Psychiatry 53, 1089-1095], in which patients show difficulty in memory recall.

Consistent with several studies highlighting the importance of dendritic spines [Jacobsen, J. S. et al. (2006) Proc. Natl. Acad. Sci. USA 103, 5161-5166] [Terry, R. D., et al. (1991) Ann. Neurol. 30, 572-580] [Ryan, T. J., et al. (2015) Science 348, 1007-1013] [Tonegawa, S. et al. (2015) Neuron 87, 918-931] in relation to memory processing, an engram-cell-specific decrease in spine density that correlated with memory deficits in early AD was observed. Natural rescue of memory recall in early AD mice required the DG engram cells in which synaptic density deficits have been restored by in vivo optical LTP protocols applied to the EC cells activated during learning. By contrast, the application of optical LTP protocols to a much wider array of excitatory EC cells projecting to the DG, which may be analogous to deep brain stimulation, did not rescue memory in AD mice. A potential explanation for this observation is that DG granule cells may contribute to a variety of memories through their partially overlapping engram cell ensembles in a competitive manner, and that activation of a large number of these ensembles simultaneously may interfere with a selective activation of an individual ensemble. Thus, activation of a more targeted engram cell ensemble may be a key requirement for effective retrieval of the specific memory, which is difficult to achieve with the current deep brain stimulation strategy. Genetic manipulations of specific neuronal populations can have profound effects on cognitive impairments of AD [Cisse, M., et al. (2011) Nature 469, 47-52]. It is here proposed that strategies applied to engram circuits can support long-lasting improvements in cognitive functions, which may provide insights and therapeutic value for future approaches that rescue memory in AD and other patients.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

1. A method of selectively producing one or both of a temporally sustained stabilization of, and increase in, dendritic spine density in one or a plurality of dentate gyrus (DG) memory engram cells in a subject, comprising:
selectively inducing high-frequency neuronal firing in one or a plurality of DG memory engram cells in the subject; in an amount effective for one or both of stabilizing and increasing dendritic spine density in the one or the plurality of DG memory engram cells, wherein the stability of, and increase in, dendritic spine density is temporally sustained.

2. The method of claim 1, wherein a means for selectively inducing the high-frequency neuronal firing comprises:
(a) expressing in one or a plurality of first cells in the subject, a stimulus-activated opsin polypeptide; wherein activating the expressed stimulus-activated opsin polypeptide selectively induces high-frequency neuronal firing in the one or the plurality of DG memory engram cells in the subject; and
(b) stimulating the stimulus-activated opsin polypeptide a first time under conditions suitable to selectively induce the high-frequency neuronal firing in the one or the plurality of DG memory engram cells, wherein the induced high-frequency neuronal firing stabilizes or increases the temporally sustained dendritic spine density in the one or the plurality of DG memory engram cells.

3. The method of claim 2, further comprising:
(c) stimulating the stimulus-activated opsin polypeptide one or more subsequent times each time under an independently selected stimulation condition of one or: (i) the same as set forth in claim 2(b) and (ii) different from those set forth in claim 2(b), wherein the selected stimulation condition is suitable to selectively induce the high-frequency neuronal firing in the one or the plurality of DG memory engram cells, the induced high-frequency neuronal firing results in one or both of: stabilizing and increasing the temporally sustained dendritic spine density in the one or more DG memory engram cells.

4-12. (canceled)

13. The method of claim 2, wherein the stimulus-activated opsin polypeptide is a light-activated opsin polypeptide.

14. The method of claim 2, wherein the stimulus-activated opsin polypeptide comprises a ChIEF polypeptide or a functional variant thereof.

15. (canceled)

16. The method of claim 2, wherein the first cell is an entorhinal cortex (EC) cell or a DG cell.

17. The method of claim 2, wherein the first cell projects to at least one DG memory engram cell in the subject.

18. The method of claim 2, wherein the stimulus-activated opsin polypeptide is expressed as part of a fusion protein.

19. The method of claim 2, further comprising delivering the stimulus-activated opsin polypeptide to the first cell in the form of a fusion protein comprising the stimulus-activated opsin polypeptide, or in the form of a vector comprising a nucleic acid sequence encoding the stimulus-activated opsin polypeptide.

20-21. (canceled)

22. The method of claim 1, wherein the selective induction of the high-frequency neuronal firing in the one or more DG memory engram cells increases the synaptic connectivity between one or more entorhinal cortex (EC) cells and one or more of the DG memory engram cells in the subject.

23. (canceled)

24. The method of claim 1, wherein the temporally sustained increase in dendritic spine density in one or more dentate gyrus (DG) memory engram cells in the subject, results in at least one of: increasing, maintaining, and slowing a reduction of a level of memory retrieval in the subject.

25. The method of claim 1, wherein the temporally sustained stabilization in dendritic spine density in one or more dentate gyrus (DG) memory engram cells in the subject, results in at least one of: increasing, maintaining, and slowing a reduction of a level of memory retrieval in the subject.

26. The method of claim 1, wherein one or both of temporally sustained stabilizing and increasing dendritic spine density in the one or the plurality of DG memory engram cells in the subject, treats a memory impairment-associated disease or condition in the subject.

27-29. (canceled)

30. The method of claim 1, wherein the subject is one or more of: at elevated risk of having Alzheimer's disease, suspected of having Alzheimer's disease, and diagnosed with Alzheimer's disease.

31-32. (canceled)

33. The method of claim 2, wherein the stimulus comprises illumination.

34-35. (canceled)

36. The method of claim 2, further comprising: modifying one or more additional treatments to the subject to treat or assist in treating the memory-impairment-associated disease or condition.

37-40. (canceled)

41. The method of claim 1, wherein inducing high-frequency neuronal firing comprises inducing long-term potentiation in the DG memory engram cells.

42. The method of claim 1, wherein the subject is a mammal.

43. (canceled)

44. The method of claim 1, wherein the subject is engaged in learning during at least a portion of the high-frequency neuronal firing.

45. (canceled)

46. A method of treating memory-impairment-associated disease or condition in a subject, the method comprising:
(a) expressing in a first cell in a subject in need of such treatment, an effective amount of a stimulus-activated opsin polypeptide; wherein activating the expressed stimulus-activated opsin polypeptide selectively induces high-frequency neuronal firing in one or more DG memory engram cells in the subject; and
(b) stimulating the stimulus-activated opsin polypeptide a first time under conditions suitable to selectively induce the high-frequency neuronal firing in the one or more DG memory engram cells, wherein the induced high-frequency neuronal firing increases a level of density of dendritic spines of the one or more DG memory engram cells; the increased density is temporally sustained in the subject; and the increased density of the dendritic spines treats the memory-impairment-associated disease or condition in the subject.

47-82. (canceled)
